(19) United States
(12) Patent Application Publication
(10) Pub. No.: US 2026/0368829 A1
(43) Pub. Date: Nov. 19, 2026

(54) RULES ENGINE FOR PROCESSING MEDICAL AND PHARMACEUTICAL CLAIMS

(71) Applicant: Nayya Health, Inc., New York, NY (US)

(72) Inventors: Courtney Marie HIGGINS, Glen Ridge, NJ (US); Chiwide WONG, Elmhurst, NY (US); Keerthana PALANI, Brooklyn, NY (US)

(73) Assignee: Nayya Health, Inc., New York, NY (US)

(21) Appl. No.: 19/679,076

(22) Filed: May 15, 2026

Related U.S. Application Data
(60) Provisional application No. 63/807,003, filed on May 16, 2025.

(51) Int. Cl. G06Q 40/08; G16H 10/60

(52) U.S. Cl. CPC G06Q 40/084; G16H 10/60

(57) ABSTRACT

A data processing system implements obtaining medical claim information and pharmaceutical claim information associated with an insured user; grouping the medical claim information and the pharmaceutical claim information into one or more medical encounters; analyzing the one or more medical encounters using a rules engine to obtain a determination whether one or more medical encounters satisfy a set of criteria associated with a reimbursement opportunity label, the set of criteria including one or more inclusion criteria that indicate one or more medical codes are required to be included the claims associated with a respective medical encounter, the set of criteria including one or more exclusion criteria indicating one or more medical codes that cannot be included one or more of the claims; and performing one or more actions responsive to the rules engine determining that the one or more medical encounters satisfy the set of criteria.

### BACKGROUND

Submission of medical and/or pharmaceutical claims to insurers is a complex process. This process can be further complicated when the insured person is covered by supplemental health insurance policies that cover expenses which are not covered by the primary health insurance policy of the insured person. For instance, the supplemental health insurance policies may cover the cost of copays, deductibles, medical expenses, pharmaceutical expenses, and/or other expenses not covered by the health insurance policy of the insured person. The insured person may have more than one supplemental health insurance plan, and the insured person often does not have a complete understanding of the coverage provided by each of these policies. Consequently, the insured user may miss opportunities to have expenses associated with medical and/or pharmaceutical claims reimbursed by their supplemental health insurance plan. Hence, there is a need for improved systems and methods that provide a technical solution for solving the technical problem of automatically analyzing medical and pharmaceutical claims and policy information to identify an insurer that is likely to cover the claims to improve reimbursement rates medical and/or pharmaceutical expenses.

### SUMMARY

An example data processing system according to the disclosure includes a processor and a memory storing executable instructions. The instructions when executed cause the processor alone or in combination with other processors to perform operations including receiving, via a network interface and over a network connection, billed event information for one or more events associated with a user, the billed event information including a plurality of electronically transmitted records encoded according to a plurality of provider-specific data formats, wherein receiving the billed event information includes parsing the plurality of electronically transmitted records to extract field values into memory; converting, by the processor, the billed event information into machine-generated unified billed information data structures conforming to a standard schema stored in the memory, wherein converting includes canonicalizing the field values extracted from the billed event information by applying one or more format-conversion routines defined by the standard schema to normalize at least identifiers, dates, and code values into corresponding normalized field values stored in the machine-generated unified billed information data structures; , by the processor, billed events represented by the machine-generated unified billed information data structures into one or more machine-generated billed event group data structures, each billed event group data structure storing references to a respective set of the machine-generated unified billed information data structures that correspond to a same billable event, wherein grouping comprises executing a deterministic rules engine to: (i) retrieve, from a grouping criteria repository stored in the memory, grouping criteria rules defining criteria for determining when to group billed events, and (ii) assign the machine-generated unified billed information data structures to the one or more machine-generated billed event group data structures based on the grouping criteria rules; retrieving, over the network connection, supplementary health insurance plan information for the user from one or more insurer portals, and storing the supplementary health insurance plan information in the memory; analyzing, by the processor using the deterministic rules engine, coverage information for each supplementary health insurance plan of the supplementary health insurance plan information to generate a plurality of reimbursement opportunity labels, wherein the deterministic rules engine includes a deterministic language model configured to (i) parse natural-language coverage descriptions included in the supplementary health insurance plan information and (ii) generate, based on results of parsing the natural-language coverage descriptions, a set of logical expression data structures that encode one or more inclusion criteria, one or more exclusion criteria, or a combination thereof, the set of logical expression data structures defining criteria including (a) one or more inclusion predicates requiring presence of specified normalized field values in one or more machine-generated unified billed information data structures referenced by a given machine-generated billed event group data structure, and (b) one or more exclusion predicates requiring absence of specified normalized field values from the one or more machine-generated unified billed information data structures referenced by the given machine-generated billed event group data structure; analyzing, by the processor using the deterministic rules engine, the one or more machine-generated billed event group data structures to perform cross-event matching by evaluating the one or more inclusion predicates and the one or more exclusion predicates represented by the set of logical expression data structures against the normalized field values stored in the machine-generated unified billed information data structures referenced by the one or more machine-generated billed event group data structures, to determine whether at least one billable event satisfies criteria associated with a reimbursement opportunity label of the plurality of reimbursement opportunity labels; and responsive to determining that the at least one billable event satisfies the criteria associated with the reimbursement opportunity label, automatically generating, by the processor, a notification message that identifies the reimbursement opportunity label and the at least one billable event, and transmitting the notification message over the network connection to a user device of the user.

An example method implemented in a data processing system includes receiving, via a network interface and over a network connection, billed event information for one or more events associated with a user, the billed event information including a plurality of electronically transmitted records encoded according to a plurality of provider-specific data formats, wherein receiving the billed event information includes parsing the plurality of electronically transmitted records to extract field values into memory; converting, by the processor, the billed event information into machine-generated unified billed information data structures conforming to a standard schema stored in the memory, wherein converting includes canonicalizing the field values extracted from the billed event information by applying one or more format-conversion routines defined by the standard schema to normalize at least identifiers, dates, and code values into corresponding normalized field values stored in the machine-generated unified billed information data structures; , by the processor, billed events represented by the machine-generated unified billed information data structures into one or more machine-generated billed event group data structures, each billed event group data structure storing references to a respective set of the machine-generated unified billed information data structures that correspond to a same billable event, wherein grouping comprises executing a deterministic rules engine to: (i) retrieve, from a grouping criteria repository stored in the memory, grouping criteria rules defining criteria for determining when to group billed events, and (ii) assign the machine-generated unified billed information data structures to the one or more machine-generated billed event group data structures based on the grouping criteria rules; retrieving, over the network connection, supplementary health insurance plan information for the user from one or more insurer portals, and storing the supplementary health insurance plan information in the memory; analyzing, by the processor using the deterministic rules engine, coverage information for each supplementary health insurance plan of the supplementary health insurance plan information to generate a plurality of reimbursement opportunity labels, wherein the deterministic rules engine includes a deterministic language model configured to (i) parse natural-language coverage descriptions included in the supplementary health insurance plan information and (ii) generate, based on results of parsing the natural-language coverage descriptions, a set of logical expression data structures that encode one or more inclusion criteria, one or more exclusion criteria, or a combination thereof, the set of logical expression data structures defining criteria including (a) one or more inclusion predicates requiring presence of specified normalized field values in one or more machine-generated unified billed information data structures referenced by a given machine-generated billed event group data structure, and (b) one or more exclusion predicates requiring absence of specified normalized field values from the one or more machine-generated unified billed information data structures referenced by the given machine-generated billed event group data structure; analyzing, by the processor using the deterministic rules engine, the one or more machine-generated billed event group data structures to perform cross-event matching by evaluating the one or more inclusion predicates and the one or more exclusion predicates represented by the set of logical expression data structures against the normalized field values stored in the machine-generated unified billed information data structures referenced by the one or more machine-generated billed event group data structures, to determine whether at least one billable event satisfies criteria associated with a reimbursement opportunity label of the plurality of reimbursement opportunity labels; and responsive to determining that the at least one billable event satisfies the criteria associated with the reimbursement opportunity label, automatically generating, by the processor, a notification message that identifies the reimbursement opportunity label and the at least one billable event, and transmitting the notification message over the network connection to a user device of the user.

An example machine-readable medium on which are stored instructions that, when executed, cause a processor alone or in combination with other processors to perform operations of receiving, via a network interface and over a network connection, billed event information for one or more events associated with a user, the billed event information including a plurality of electronically transmitted records encoded according to a plurality of provider-specific data formats, wherein receiving the billed event information includes parsing the plurality of electronically transmitted records to extract field values into memory; converting, by the processor, the billed event information into machine-generated unified billed information data structures conforming to a standard schema stored in the memory, wherein converting includes canonicalizing the field values extracted from the billed event information by applying one or more format-conversion routines defined by the standard schema to normalize at least identifiers, dates, and code values into corresponding normalized field values stored in the machine-generated unified billed information data structures; , by the processor, billed events represented by the machine-generated unified billed information data structures into one or more machine-generated billed event group data structures, each billed event group data structure storing references to a respective set of the machine-generated unified billed information data structures that correspond to a same billable event, wherein grouping comprises executing a deterministic rules engine to: (i) retrieve, from a grouping criteria repository stored in the memory, grouping criteria rules defining criteria for determining when to group billed events, and (ii) assign the machine-generated unified billed information data structures to the one or more machine-generated billed event group data structures based on the grouping criteria rules; retrieving, over the network connection, supplementary health insurance plan information for the user from one or more insurer portals, and storing the supplementary health insurance plan information in the memory; analyzing, by the processor using the deterministic rules engine, coverage information for each supplementary health insurance plan of the supplementary health insurance plan information to generate a plurality of reimbursement opportunity labels, wherein the deterministic rules engine includes a deterministic language model configured to (i) parse natural-language coverage descriptions included in the supplementary health insurance plan information and (ii) generate, based on results of parsing the natural-language coverage descriptions, a set of logical expression data structures that encode one or more inclusion criteria, one or more exclusion criteria, or a combination thereof, the set of logical expression data structures defining criteria including (a) one or more inclusion predicates requiring presence of specified normalized field values in one or more machine-generated unified billed information data structures referenced by a given machine-generated billed event group data structure, and (b) one or more exclusion predicates requiring absence of specified normalized field values from the one or more machine-generated unified billed information data structures referenced by the given machine-generated billed event group data structure; analyzing, by the processor using the deterministic rules engine, the one or more machine-generated billed event group data structures to perform cross-event matching by evaluating the one or more inclusion predicates and the one or more exclusion predicates represented by the set of logical expression data structures against the normalized field values stored in the machine-generated unified billed information data structures referenced by the one or more machine-generated billed event group data structures, to determine whether at least one billable event satisfies criteria associated with a reimbursement opportunity label of the plurality of reimbursement opportunity labels; and responsive to determining that the at least one billable event satisfies the criteria associated with the reimbursement opportunity label, automatically generating, by the processor, a notification message that identifies the reimbursement opportunity label and the at least one billable event, and transmitting the notification message over the network connection to a user device of the user.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

The drawing figures depict one or more implementations in accord with the present teachings, by way of example only, not by way of limitation. In the figures, like reference numerals refer to the same or similar elements. Furthermore, it should be understood that the drawings are not necessarily to scale.

FIG. 1A is a diagram of an example reimbursement opportunity pipeline in which the techniques for identifying a supplemental insurance policy that may cover medical and/or pharmaceutical claims herein are implemented.

FIG. 1B is a diagram of an example implementation of the rules engine of the reimbursement opportunity pipeline shown in FIG. 1A.

FIG. 2 is a diagram showing an example computing environment that includes a benefits service platform that utilizes the reimbursement opportunity pipeline shown in FIG. 1A.

FIG. 3 is a diagram of an example user interface of the employee portal shown in FIG. 2.

FIGS. 4A, 4B, 4C, 4D, and 4E, are diagrams showing example reimbursement opportunities according to the techniques herein.

FIGS. 5A and 5B are a flow chart of an example process for identifying reimbursement opportunities in which medical and/or pharmaceutical claims according to the techniques disclosed herein.

FIG. 6 is a block diagram showing an example software architecture, various portions of which may be used in conjunction with various hardware architectures herein described, which may implement any of the described features.

FIG. 7 is a block diagram showing components of an example machine configured to read instructions from a machine-readable medium and perform any of the features described herein.

### DETAILED DESCRIPTION

Systems and methods are provided for analyzing medical and/or pharmaceutical claims associated with an insured user and determining whether a supplemental insurance policy of the insured user will cover the medical claims and/or pharmaceutical claims. Various types of supplemental insurance policies are offered that provide various types of coverage. For instance, examples of supplemental policies can include but are not limited to dental insurance, vision insurance, hospital indemnity insurance, critical illness insurance, accident insurance, disability insurance, short-term health insurance, supplemental insurance for Medicare, or insurance that provides coverage for specific diseases.

The present application provides a reimbursement opportunity pipeline that provides a technical solution for automatically analyzing medical and pharmaceutical claims and supplemental insurance policy information to identify an insurer that is likely to cover the claims to improve reimbursement rates medical and/or pharmaceutical expenses. The reimbursement opportunity pipeline implements a rules engine that analyzes the medical claims and/or pharmaceutical claims and groups the claims into medical encounters to perform cross-claim analysis of the claims included in the group. A medical encounter, as used herein, refers to interactions between one or more healthcare providers and the insured user for the purpose of providing healthcare services to the insured user and/or assessing the health of the insured user. A medical encounter can be associated with multiple medical claims and/or pharmaceutical claims depending on the nature of the medical encounter.

The rules engine then performs cross-claim analysis on the group of claims associated with each medical encounter to determine whether these claims satisfy reimbursement opportunity criteria associated with the supplemental insurance policies of the insured user. The reimbursement opportunity criteria can include inclusion criteria and/or exclusion criteria. The inclusion criteria indicate one or more medical codes are required to be included on one or more of the medical claims or pharmaceutical claims associated with a respective medical encounter. The exclusion criteria indicate one or more medical codes that cannot be included one or more of the medical claims or pharmaceutical claims. The inclusion of a medical code associated with an exclusion criterion indicates that the medical and/or pharmaceutical claims associated with the medical encounter are excluded from reimbursement according to the benefits defined by the plan certificate of the supplemental insurance policy. The cross-claim analysis performed by the rules engine provided herein analyzes claims the claims within the group as a whole rather than individually analyzing claims to determine whether the claims satisfy the including and/or exclusion criteria for the supplemental insurance policy. A technical benefit of this approach is that the rules engine is more likely to correctly predict whether a supplemental insurance policy will reimburse medical and/or pharmaceutical claims, thereby improving reimbursement rates medical and/or pharmaceutical expenses. Another technical benefit of this approach is that this approach can significantly improve efficiency and reduce computing resources associated with processing medial and/or pharmaceutical claims by reducing the likelihood that users will need to resubmit claims multiple times for processing due to errors in processing claims associated with analyzing claims on a claim-by-claim basis.

An insured user may have multiple supplemental insurance policies, and it is possible that one policy may include exclusion criteria that preclude the reimbursement of claims associated with a particular medical encounter. The insured user may not be aware of what is and what is not covered by their supplemental insurance policies and may easily miss out on reimbursement opportunities, because another of their supplemental insurance policies may cover the claims that were excluded by another supplemental insurance policy. The rules engine analyzes all of the health insurance policies associated with the user to ensure that no such reimbursement opportunities are overlooked. This approach provides a technical solution to the technical problem associated with identifying reimbursement opportunities. A technical advantage of the rules engine provided herein is that the rules engine utilizes rules to clearly define which payment opportunities are available, including cross-referencing information across medical and/or pharmaceutical claims, and does not rely on computationally expensive artificial intelligence models to make such determinations. Furthermore, the rules engine provides a deterministic output compared to the use of artificial intelligence models. The rules engine provides a predictable and transparent solution for processing claims that can be easily audited to ensure compliance with relevant industry and/or regulatory requirements. In contrast, artificial intelligence models are probabilistic and may return different results even when reasoning over the same set of data. Artificial intelligence models can also be subject to hallucinations in which the model produces factually incorrect inferences, which may cause the model to incorrectly infer that a set of claims should or should not be reimbursed for an insured user. These and other technical advantages of the techniques disclosed herein will be evident from the discussion of the example implementations that follow.

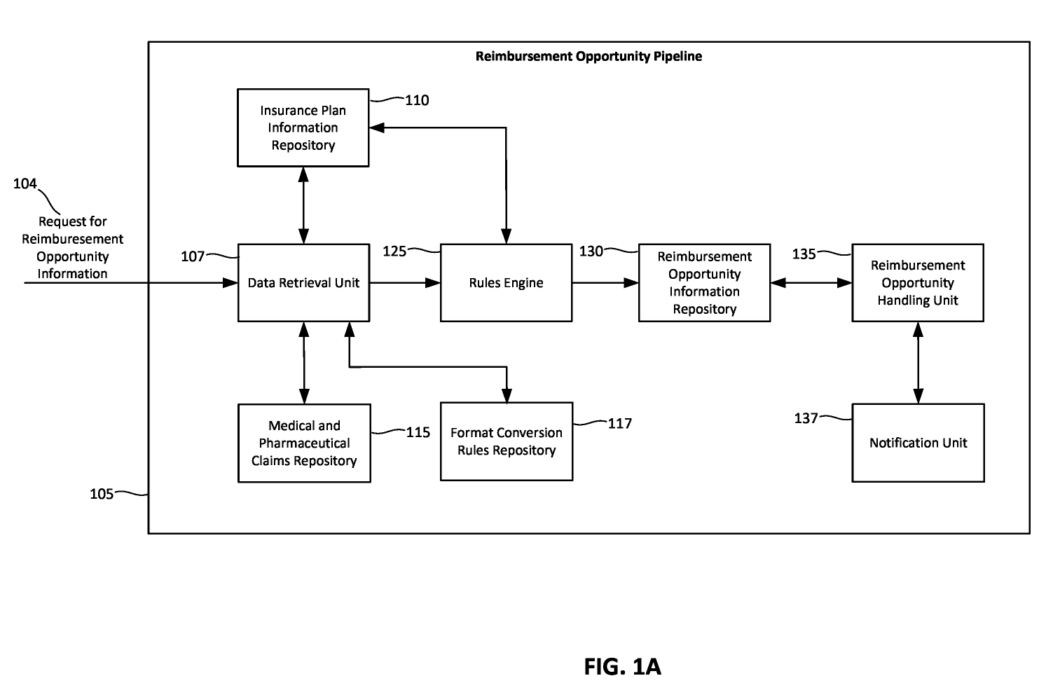

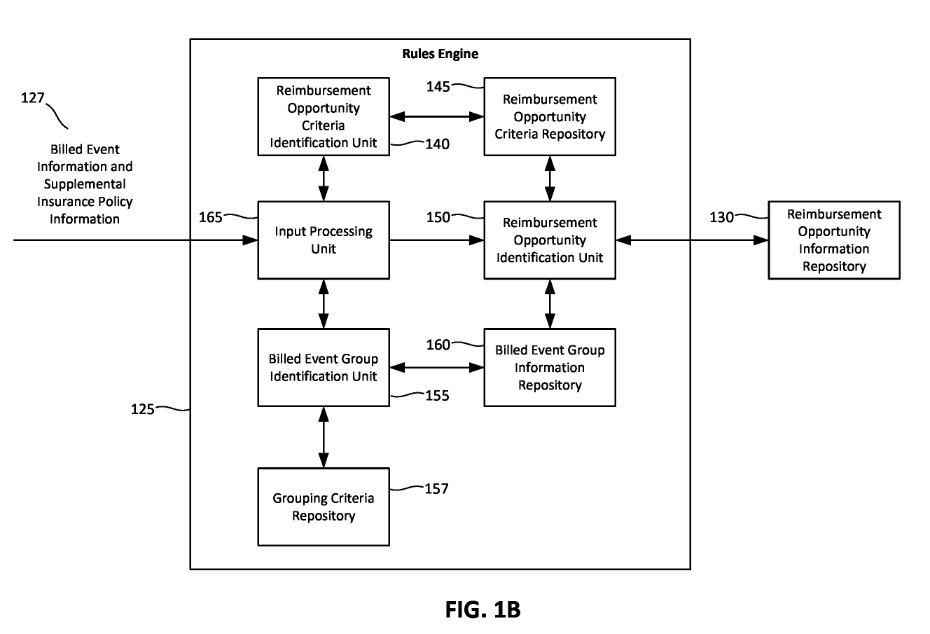

FIG. 1A is a diagram of an example reimbursement opportunity pipeline 105 in which the techniques for identifying a supplemental insurance policy that may cover medical and/or pharmaceutical claims herein are implemented. The reimbursement opportunity pipeline 105 receives a request to generate reimbursement opportunity information 104 that identifies an insured user for which a supplemental health insurance plan may pay expenses associated with one or more medical claim and/or pharmaceutical claims associated with the insured user. The request to generate the reimbursement opportunity information 104 can originate from an employee portal of a benefit services platform. The employee portal enables the insured user to view benefits information for employer provided benefits and provides tools that enable the insured user to learn about and to utilize their benefits. The employee portal can include a user interface that enables the insured user to view medical and/or pharmaceutical claims that have not been reimbursed by their primary insurance policies and to analyze these claims to identify reimbursement opportunities where a supplemental insurance policy of the insured user is likely to reimburse all or at least a portion of the claims that were not reimbursed by the primary insurance policy. The request to generate reimbursement opportunity information 104 can also originate from a service that operates on the benefits service platform that automatically submits requests on behalf of insured users who have accounts with the benefit service platform to proactively identify reimbursement opportunities for these insured users. The service can be configured to automatically check periodically whether insured users have new medical claims and/or pharmaceutical claims that were not reimbursed in full or in part by the primary insurance of the insured users.

The data retrieval unit 107 receives the request and accesses the insurance plan information repository 110 in response to receiving the request. The data retrieval unit 107 obtains policy information for supplemental health insurance plans for the insured user from the insurance plan information repository 110. The insurance plan information repository 110 is a persistent data store that stores supplemental health insurance plan information for multiple insured users. The insured user may be covered by one or more supplemental health insurance plans. Each of these supplemental health insurance plans can provide coverage for different types of medical and/or pharmaceutical claims. As will be discussed in greater detail in the examples which follow, the supplemental health insurance plans can provide inclusion criteria that define the types of medical and/or pharmaceutical claims that may be reimbursed by the insurer. The supplemental health insurance plans can also provide exclusion criteria that define specific circumstances under which the insurer will not reimburse medical and/or pharmaceutical claims of the insured user.

The insurance plan information repository 110 can include a policy information data structure for storing supplemental health insurance policy information. The policy information data structure can include fields for storing a unique identifier associated with an insured user and a unique identifier associated with a supplemental insurance plan that provides coverage with the insured user. The policy information data structure can also include additional information such as but not limited to an effective date and/or expiry date of the supplemental insurance plan. The insurance plan information repository 110 can include a policy coverage data structure that includes coverage information from the insurance policy. The coverage information extracted from the insurance policy can include inclusion and/or exclusion criteria that are expressed in natural language that explain what is and what is not covered by the supplemental insurance policy. The coverage information may be expressed in various parts of the policy document, such as but not limited to a declarations section, an exclusion section, an endorsement section, and/or other sections of the electronic copy of the supplemental insurance policy obtained from the insurance plan information repository 110.

The data retrieval unit 107 also obtains billed event information for one or more events associated with the user. The billed event information the billed event information includes a plurality of electronically transmitted records encoded according to a plurality of provider-specific data formats. The data retrieval unit 107 parses the plurality of electronically transmitted records to extract field values into a memory allocated to the reimbursement opportunity pipeline 105.

The billed event information can be medical and/or pharmaceutical claims. The data retrieval unit 107 can medical and/or pharmaceutical claims associated with the insured user from the medical and pharmaceutical claims repository 115 in response to receiving the request to generate reimbursement opportunity information 104. A medical claim, as used herein, refers to a request for payment that a healthcare provider submits to the insurer providing health insurance for an insured user. Pharmaceutical claims, as used herein, refer to a request for payment for pharmaceuticals that have been prescribed to the insured user. The pharmaceutical claims may be submitted to the insurer by a pharmacy or by the medical service provider. The medical and/or pharmaceutical claims that are obtained by the data retrieval unit 107 are medical and/or pharmaceutical claims that have either not been reimbursed by the primary health insurance of the insured user or have only been reimbursed a portion of the total cost associated with medical and/or pharmaceutical claims. The reimbursement opportunity pipeline 105 analyzes these claims and attempts to identify reimbursement opportunities where one or more of the supplemental health insurance plans for the insured user will reimburse at least a portion of the costs associated with the medical and/or pharmaceutical claims that have not been reimbursed by the primary health insurance of the insured user.

The data retrieval unit 107 provides the supplemental insurance information obtained from the insurance plan information repository 110 and the billed event information (which can be medical and/or pharmaceutical claim information obtained from the medical and pharmaceutical claims repository 115) to the rules engine 125. The rules engine 125 is a deterministic rules engine that groups the billed event information into billed event groups. In implementations in which the billed event information represents medical claims and/or pharmaceutical claims, the rules engine groups the medical claims and/or pharmaceutical claims into billed event groups referred to as medical encounters. The rules engine 125 also extracts inclusion and exclusion criteria from the supplemental health insurance plan information, and applies the inclusion and exclusion criteria to the billed event information to determine reimbursement opportunity information to be output to the reimbursement opportunity information repository 130. Additional details of the rules engine 125 shown in FIG. 1B, which is discussed in detail below.

The data retrieval unit 107 can be configured to convert the billed event information into machine-generated unified billed information data structures conforming to a standard schema stored in a memory of the reimbursement opportunity pipeline 105. The standard schema represents a set of logical data structures that may be used by the reimbursement opportunity pipeline 105 storing and analyzing data. The standard schema can also define format-conversion routines for normalizing data in the billed event information to a normalized format. Converting the billed event information into the into machine-generated unified billed information data structures includes canonicalizing the field values extracted from the billed event information by applying one or more format-conversion routines defined by the standard schema to normalize at least identifiers, dates, and code values into corresponding normalized field values stored in the machine-generated unified billed information data structures. This canonicalization process is necessary in some implementations because the billed event information can be encoded according to a variety of different provider-specific data formats.

In implementations in which the billed event information includes medical claim and/or pharmaceutical claim information obtained from the medical and pharmaceutical claims repository 115 the data retrieval unit 107 can canonicalize the medical claim and/or pharmaceutical claim information. For instance, the standard schema can define a unified claim data structure that can include normalized fields for medical codes, dates of services, provider information, and/or other information associated with the medical claims and/or pharmaceutical claims that has been normalized according to the standard format defined by the standard schema. The medical claim and/or pharmaceutical claim information obtained from different providers may not be formatted consistently and may be received in a plurality of heterogeneous data formats. The data retrieval unit 107 can utilize mapping rules to map the medical claim and/or pharmaceutical claim information into a unified claim data structure conforming to the standard schema.

The data retrieval unit 107 can also convert the supplemental insurance information obtained from the insurance plan information repository 110 according to the standard schema. The schema can define a unified supplemental insurance information policy data structure. The supplemental insurance information may also be provided in a plurality of heterogeneous formats by insurers and canonicalizing this information using the standard scheme enables the rules engine 125 to operate policy information having a consistent format. The data retrieval unit 107 can utilize mapping rules to map the supplemental insurance information received from the providers into the unified supplemental insurance policy data structure in which the policy information has been normalized according to the standard schema.

This approach provides a technical solution to the technical problems associated implementing such rules in executable program code. A technical advantage of this approach is that the mapping rules can be stored in a format conversion rules repository 117 and the data retrieval unit 107 can access and execute these rules on the billed event information, the medical claim and/or pharmaceutical claim information, and/or the supplemental insurance policy information. Another technical advantage of this approach is that the rules for a particular data source can be updated and deployed rapidly without requiring the program code that implements the data retrieval unit 107 to be updated and the software redeployed. Furthermore, converting the claims data to the standard schema also enables the deterministic rules engine to operate on medical claim and/or pharmaceutical claim information that has a consistent format. Another technical advantage is that this approach significantly reduces the complexity of the rules engine because the engine does not have to be adapted to handle data in numerous different formats that may be changed by the providers of this data. Consequently, the rules engine will require less complex code that will require fewer software updates, which enables the deterministic rules engine to operate in a less computationally intensive manner. Another technical benefit of this approach is that the reduced complexity of the deterministic rules engine is significantly less complex than an artificial intelligence model and requires significantly fewer computing resources to operate than an artificial intelligence model configured to perform similar tasks as those performed by the deterministic rules engine. Consequently, the deterministic rules engine can be operated on hardware that has lower power and cooling requirements that that used to implement an artificial intelligence model. For instance, artificial intelligence models are typically implemented on Graphics Processing Units (GPUs) which comprise specialized, parallel processors that are designed for high throughput applications, while the deterministic rules engine disclosed herein can be implemented on less expensive and less resource intensive Central Processing Units (CPUs). Another benefit of this approach is that the rules engine may be operated on smaller, resource constrained devices rather than requiring deployment to a cloud-based computing environment.

The reimbursement opportunity information generated by the rules engine 125 identifies one or more of the supplemental health insurance policies of the insured user that may reimburse at least a portion of the costs associated with the billed event information, which may include medical claims and/or pharmaceutical claims associated with one or more medical encounters. The reimbursement opportunity information can be stored in a reimbursement opportunity information repository 130 which is implemented in a persistent memory allocated to the reimbursement opportunity pipeline 105, and the reimbursement opportunity information be formatted according to a reimbursement opportunity data structure that associates the reimbursement opportunity with an insured user and supplemental health insurance policy. The reimbursement opportunity information can also include information identifying any exclusions associated with the billed event information that preclude one or more of the insurers from reimbursing at least a portion of the costs associated with the billed event information. For instance, the reimbursement opportunity information can include information identifying exclusions associated with one or more medical encounters that preclude one or more of the insurers from reimbursing at least a portion of the costs associated with the medical claims and/or pharmaceutical claims associated with one or more medical encounters.

The opportunity handling unit 135 can perform various actions on the reimbursement opportunity information stored in the reimbursement opportunity information repository 130. For instance, the opportunity handling unit 135 can signal the notification unit 137 to automatically generate a notification to the insured user of the opportunity to obtain reimbursement under one or more of their supplemental insurance policies. The notification can be sent via text message, email, a physical letter, a notification on a benefits portal, and/or via other means. The notification can include instructions that indicate how to submit the medical and/or pharmaceutical claims to an insurer and/or for authorizing a benefits service platform to automatically submit the claims on behalf of the insured user. The opportunity handling unit 135 can also automatically submit the claims on behalf of the insured user where the insured user has provided consent to perform such actions behalf of the insured user.

FIG. 1B is a diagram of an example implementation of the rules engine 125 of the reimbursement opportunity pipeline 105 shown in FIG. 1A. The input processing unit 165 receives the machine-generated unified billed information data structures representing the billed event information and the unified supplemental insurance information policy data structures representing the supplemental insurance policies associated with the insured user. The billed event information can comprise unified claim data structures that represent medical and/or pharmaceutical claims information. The input processing unit 165 provides the billed event information to the billed event group identification unit 155 for processing and the supplemental insurance policy information to the reimbursement opportunity criteria identification unit 140 for processing.

The billed event group identification unit 155 groups the billed event information into billed event groups which are stored in machine-generated billed event group data structures. Each billed event group data structure stores references to a respective set of the machine-generated unified billed information data structures that correspond to a same billable event. The billed event group identification unit 155 of the deterministic rules engine performs operations to: (i) retrieve, from a grouping criteria repository 157 stored in the memory allocated to the reimbursement opportunity pipeline 105, grouping criteria rules defining criteria for determining when to group billed events, and (ii) assign the machine-generated unified billed information data structures to the one or more machine-generated billed event group data structures based on the grouping criteria rules. The billed event group identification unit 155 stores the machine-generated billed event group data structures in the billed event group information repository 160.

In implementations in which the billed event group information comprises machine-generated unified billed information data structures, the billed event group identification unit 155 groups the machine-generated unified billed information data structures representing medical and/or pharmaceutical claims by medical encounter which are stored in a medical encounter data structure. A medical encounter refers to a unique medical event that can include providing healthcare services and/or assessing the health of a patient. A medical encounter may be associated with one or more medical claims associated with medical services provided and/or one or more pharmaceutical claims for medications prescribed to the insured user. The medical encounter data structure can include a unique identifier associated with the unified claim structure associated with each of the medical claims and/or pharmaceutical claims associated with the medical encounter. Grouping the medical claims and/or pharmaceutical claims into a medical encounter data structure provides a technical solution to the problems associated with accurately identifying reimbursement opportunities, because the information used to determine whether the inclusion criteria and/or the exclusion criteria associated with a reimbursement opportunity are often distributed across multiple medical claims and/or pharmaceutical claims. Current approaches for identifying reimbursement opportunities typically assess the medical claims and/or pharmaceutical claims individually. However, individually assessing claims is error prone and can often result in an erroneous determination that the inclusion criteria and/or the exclusion criteria were not being satisfied when a correct assessment would have been made had multiple claims associated with a medical encounter been analyzed as a group. A technical advantage of grouping medical and/or pharmaceutical claims into medical encounters helps to improve the identification of reimbursement opportunities as well as exclusions.

The billed event group identification unit 155 can group the medical and/or pharmaceutical claims using various criteria. The criteria can be stored as grouping criteria rules in the grouping criteria repository 157, which is implemented in a persistent memory allocated to the rules engine 125 of the reimbursement opportunity pipeline 105. The grouping criteria rules can be stored as grouping criteria rules data structures in the grouping criteria repository 157. The grouping criteria can be defined by an authorized user and may be periodically updated as changes to medical and/or pharmaceutical claim coding are identified. The rules engine 125 can provide a user interface that enables authorized users to define grouping criteria, to modify existing grouping criteria, and/or to delete existing grouping criteria.

The following non-limiting provide some examples of the types of grouping criteria rules that may be stored in the grouping criteria repository 157 and used by the billed event group identification unit 155 to group medical and/or pharmaceutical claims. For instance, in some implementations, the billed event group identification unit 155 groups the medical and/or pharmaceutical claims based on the start and end dates of the claims. For instance, in a non-limiting example, the start and end dates for a medical encounter related to hospitalization would be the date of admission and the date of discharge from the hospital. The billed event group identification unit 155 groups the medical and/or pharmaceutical claims based on the place of service and/or the National Provider Identifier (NPI) or other unique identifier for the healthcare provider. Another technical benefit of grouping the medical and/or pharmaceutical claims into medical encounters can reduce the computational resources utilized when processing claims by significantly reducing and/or eliminating errors associated with reimbursement that would cause users to resubmit claims multiple times for processing. The billed event group identification unit 155 outputs the grouped claims as medical encounter data structures and stores the medical encounter data structures in the billed event group information repository 160. The billed event group information repository 160 is implemented in a persistent memory allocated to the rules engine 125 of the reimbursement opportunity pipeline 105. The medical encounter data structure can include a unique encounter identifier associated with the group of claims and a claim identifier associated with each of the medical and/or pharmaceutical claims associated with the claim group. While the preceding example discusses billed events and billed events groups related to medical and/or pharmaceutical claims, the reimbursement opportunity pipeline 105 is not limited to medical and/or pharmaceutical claims. Other types of billing events and billing event groups can be utilized in other implementations.

The reimbursement opportunity criteria identification unit 140 analyzes the supplemental insurance policy information for the supplemental insurance policies associated with the insured user and outputs reimbursement opportunity criteria which are stored in the reimbursement opportunity criteria repository 145. The reimbursement opportunity criteria repository 145 is implemented in a persistent memory allocated to the rules engine 125 of the reimbursement opportunity pipeline 105. The supplemental insurance policy information indicates what is and what is not covered by each of the supplemental insurance policies. The reimbursement opportunity criteria identification unit 140 can analyze the policy coverage data structures that store the policy coverage information to identify exclusion and/or inclusion criteria defined in the policy coverage information. As discussed above, the policy coverage information can be expressed as natural language in the supplemental insurance policy information stored in the insurance plan information repository 110. The rules engine can implement deterministic, non-AI-based language model that is configured to (i) parse natural-language coverage descriptions included in the supplementary health insurance plan information and (ii) generate, based on results of parsing the natural-language coverage descriptions, a set of logical expression data structures that encode one or more inclusion criteria, one or more exclusion criteria, or a combination thereof. The deterministic language model can output the set of logical expressions as a set of logical expression data structures that include a policy identifier and one or more logical expressions representing the including criteria and/or exclusion criteria extracted from the natural language description. The logical expressions can define inclusion criteria as one or more inclusion predicates requiring presence of specified normalized field values in one or more machine-generated unified billed information data structures referenced by a given machine-generated billed event group data structure, and exclusion criteria as one or more exclusion predicates requiring absence of specified normalized field values or a combination thereof from the one or more machine-generated unified billed information data structures referenced by the given machine-generated billed event group data structure. The deterministic language model can be implemented using a language model that utilizes a rules-based and/or pattern matching approach to identify and extract relevant portions of the policy declarations.

In implementation in which the billed events are medical and/or pharmaceutical claims, the exclusion and/or inclusion criteria can be represented as a set of medical codes that must be present in the medical and/or pharmaceutical claims to satisfy either the inclusion criteria or the exclusion criteria. The medical codes can be combined together in complex Boolean logic expressions that enable the inclusion criteria and/or the exclusion criteria to express various combinations of medical codes that can satisfy the inclusion criteria and/or the exclusion criteria. Additional examples of such inclusion and exclusion criteria are discussed in detail in the examples which follow. A technical benefit of this approach is that the language model can utilize a set of rules and/or patterns that can be updated without requiring executable program code of the model to be updated. Furthermore, the model can be implemented without utilizing an AI-based language model, which enables the reimbursement opportunity criteria identification unit 140 to be implemented in resource constrained computing environments, which AI-based language model typically require significantly more computing resources as well as more expensive Graphics Processing Units (GPUs) that facilitate complex parallel processing tasks.

The reimbursement opportunity criteria identification unit 140 generates reimbursement opportunity criteria from the logical expressions output by the deterministic language model. The reimbursement opportunity criteria groups the logical expression data structures extracted from the supplemental insurance policy information and stores the reimbursement opportunity criteria as a reimbursement criteria data structure in the The reimbursement opportunity criteria are used by the rules engine 125 to determine whether the medical claims and/or pharmaceutical claims associated with a particular medical encounter may be reimbursed by an insurer providing a supplemental insurance policy of the insured user. The reimbursement criteria can be stored in a reimbursement criteria data structure that associates one or more exclusion criteria and/or one or more inclusion criteria extracted from the policy declarations with the unique identifier associated with a respective supplemental insurance plan that provides coverage with the insured user. The respective supplemental insurance plan can be associated with multiple exclusion criteria and/or inclusion criteria entries depending upon how many exclusion criteria and/or inclusion criteria are defined in the policy for the supplemental health insurance plan.

The reimbursement opportunity criteria represent logical groupings of reimbursement criteria referred to herein as reimbursement opportunity labels. Each reimbursement opportunity label is associated with a particular type of medical encounter for which the insurer will reimburse at least a portion of the costs associated with the medical claims and/or pharmaceutical claims associated with the medical encounter. For instance, in a non-limiting example, a diabetes label is associated with a particular set of medical codes that are associated with the insured user receiving treatment for diabetes, while a heart attack label is associated with a particular set of medical codes associated with the insured user experiencing a heart attack.

Each reimbursement opportunity label is associated with one or more inclusion criteria. The reimbursement opportunity label may also be associated with one or more exclusion criteria. The one or more inclusion criteria define a trigger condition, which if satisfied, indicates that at least a portion of the costs associated with the claims associated with a medical encounter may be reimbursed by a supplemental insurance policy of the insured user as long as no exclusion criteria associated with the reimbursement opportunity label have been satisfied. The inclusion criteria define one or more medical codes are required to be included on one or more of the medical claims or pharmaceutical claims associated with a respective medical encounter to trigger the reimbursement opportunity. The exclusion criteria indicate one or more medical codes, which if included on a claim associated with a medical encounter, will result in the insurer declining to reimburse the medical and/or pharmaceutical claims associated with the medical encounter even if the inclusion criteria have been satisfied. The inclusion criteria can include logic that defines combinations of medical codes on the medical claims and/or pharmaceutical claims associated with the medical encounter that trigger the reimbursement opportunity label. Similarly, the exclusion criteria can include logic that defines combinations of medical codes which, if included on the medical claims and/or pharmaceutical claims associated with the medical encounter, result in the insurer declining to reimburse the claims. Embedding logic in the inclusion criteria and/or exclusion criteria enables the rules engine 125 to match various medical coding scenarios that can represent a particular type of medical event covered by a supplemental insurance policy of the user. Medical providers can take different approaches to medical coding that could result in reimbursement opportunities being overlooked. The embedded logic utilized herein provides a flexible solution to addressing such coding inconsistencies that improve the accuracy and the performance of the rules engine 125.

The following non-limiting example illustrates how the reimbursement opportunity criteria and the reimbursement opportunity labels can be defined. In this example, a supplemental health insurance policy reimburses medical and/or pharmaceutical claims if the user experiences a heart attack. The reimbursement opportunity criteria identification unit 140 extracts the reimbursement opportunity label information from the supplemental insurance policy information to create a heart attack label that includes the inclusion criteria that triggers the reimbursement opportunity for a medical encounter resulting from the insured user being treated for a heart attack. In this non-limiting example, the inclusion criteria is defined as: ((Diagnosis Code A AND Procedure Code B) OR Diagnosis-Related Group C). The inclusion criteria logic requires that: (1) that both Diagnosis Code A and Procedure Code B be present one or more of the medical and/or pharmaceutical claims associated with the medical encounter or (2) the Diagnosis-Related Group (DRG) indicator C is present on one or more of the medical and/or pharmaceutical claims associated with the medical encounter. The specific medical codes that are included in the inclusion and/or exclusion criteria output by the reimbursement opportunity criteria identification unit 140 are determined based on the policy limitations included in the supplemental insurance policy information. As will be discussed in detail below, the specific codes included in the inclusion criteria and/or the exclusion criteria are also mapped to a suite of overlapping medical codes that represent equivalent indicators of an occurrence of a particular medical event to improve the likelihood that the reimbursement opportunity pipeline 105 will identify a potential reimbursement opportunity even when the claims information omits certain medical code information.

The reimbursement opportunity identification unit 150 analyzes the medical encounter information stored in the billed event group information repository 160 and the reimbursement opportunity criteria stored in the reimbursement opportunity criteria repository 145 to determine whether the billed event group included in the medical encounter information of the billed event group information repository 160 are eligible for coverage by one or more of the supplemental insurance policies of the insured user. The reimbursement opportunity identification unit 150 performs cross-event matching in which by evaluating the one or more inclusion predicates and the one or more exclusion predicates represented by the set of logical expression data structures against the normalized field values stored in the machine-generated unified billed information data structures referenced by the one or more machine-generated billed event group data structures, to determine whether at least one billable event satisfies criteria associated with a reimbursement opportunity label of the plurality of reimbursement opportunity labels.

In instance in which the billed events are represented by medical and/or pharmaceutical claims associated with a medical encounter, the reimbursement opportunity identification unit 150 determines whether one or more exclusion predicates associated with exclusion criteria and/or one or more inclusion predictions associated with inclusion predicates are satisfied by the normalized field values included in the unified claim data structures associated with the medical and/or pharmaceutical claims. As discussed above, cross-event matching significantly improves the reimbursement determinations made by the reimbursement opportunity identification unit 150 by considering the medical codes across medical and/or pharmaceutical claims for which the user has been billed rather than individually considering such claims in isolation. This approach can significantly improve the reimbursement determinations, because information required to make an accurate determination is often distributed across multiple claims associated with a medical encounter.

To improve reimbursement matching, the reimbursement opportunity identification unit 150 maps the medical codes included in the inclusion criteria and/or the exclusion criteria of the reimbursement opportunity labels to a robust set of medical codes and fields from the claims. A technical problem associated with identifying reimbursement opportunities is that certain medical providers may omit certain medical codes from the medical claims and/or pharmaceutical claims, but the reimbursement opportunity identification unit 150 can instead rely on medical codes from alternative fields or combinations of fields from the claims that provide equivalent information. The reimbursement opportunity identification unit 150 provides a technical solution to this problem by mapping the medical codes to a robust set of medical codes and fields from the claims. A technical advantage of this approach is that it significantly increases the likelihood of the reimbursement opportunity identification unit 150 identifying a valid reimbursement opportunity in view of inconsistent approaches to medical coding taken by medical providers. In a non-limiting example to illustrate this concept, when the CPT code associated with one or more medical claims associated with a medical encounter is withheld or accidentally omitted, the reimbursement opportunity identification unit 150 can rely on equivalent information such as the ICD diagnosis code and/or the DRG indicator to determine whether the medical claims satisfy the inclusion and/or exclusion criteria for a particular reimbursement opportunity label.

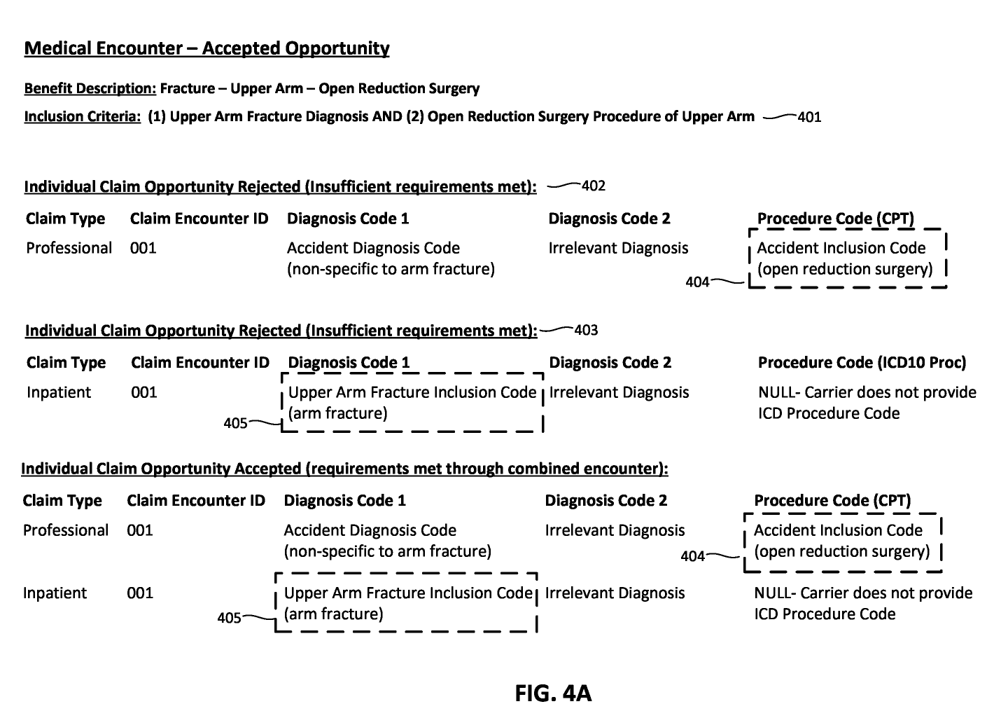

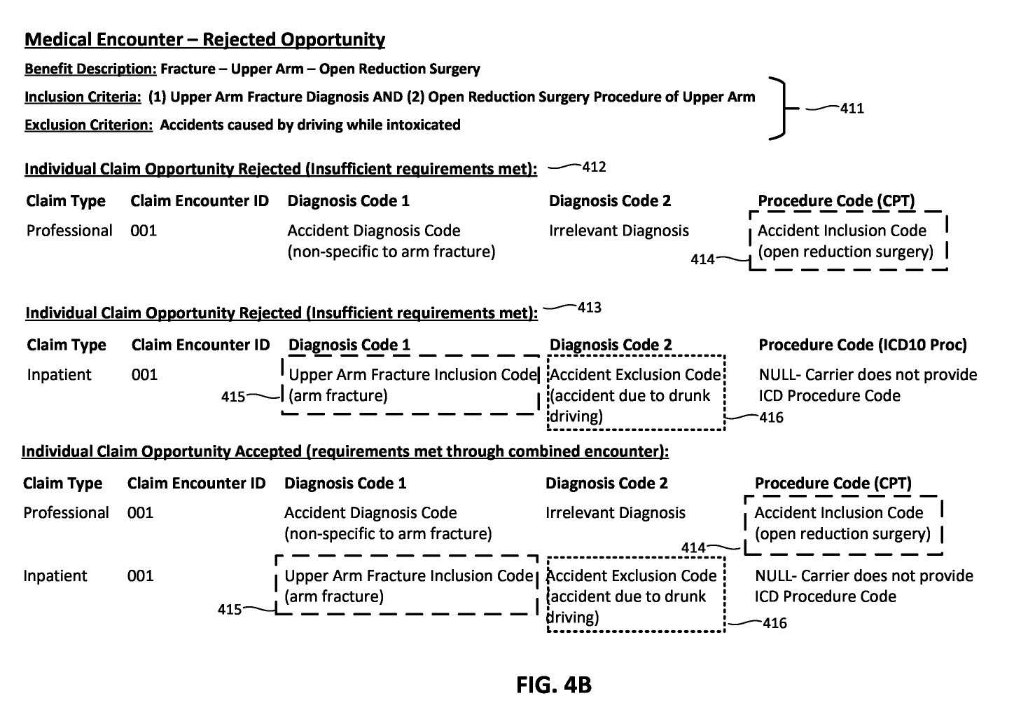

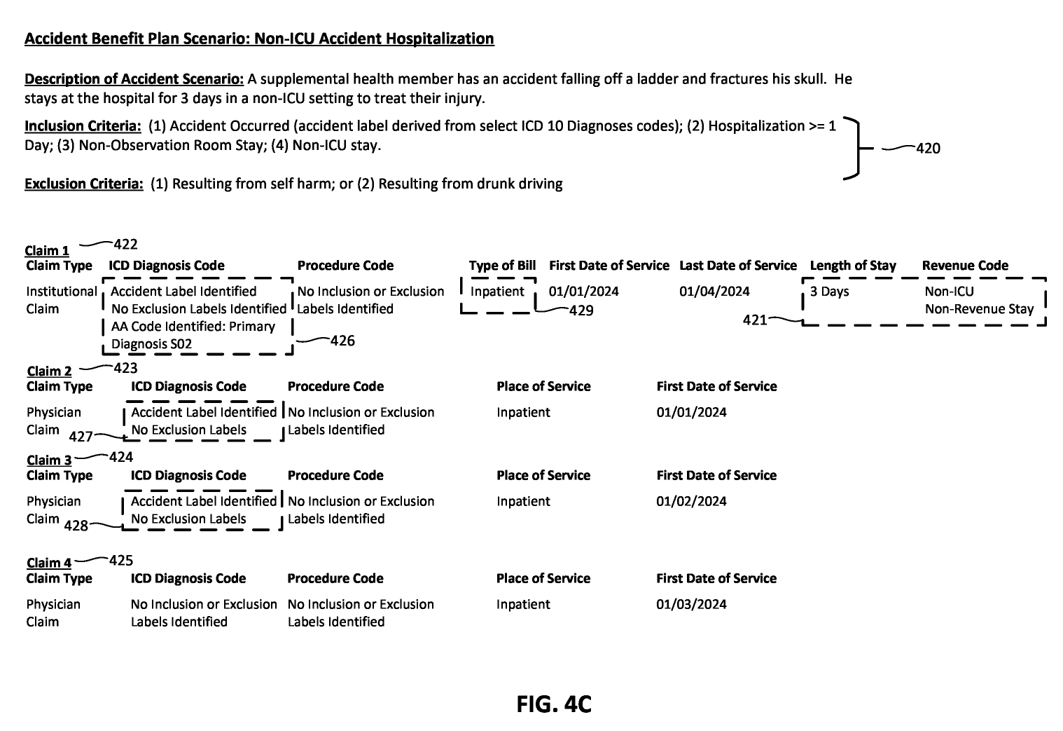

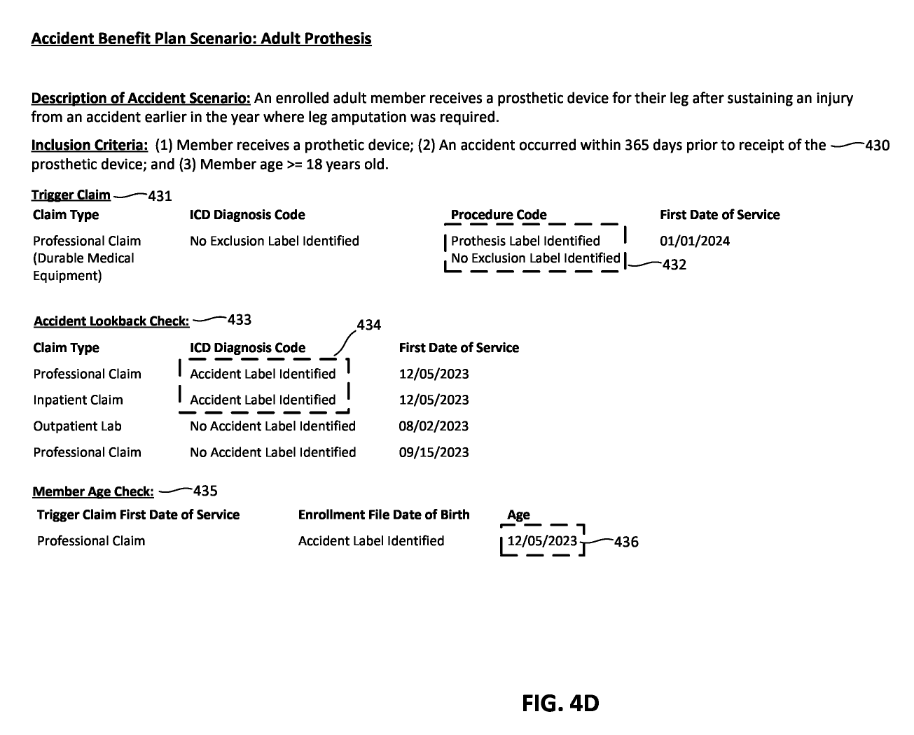

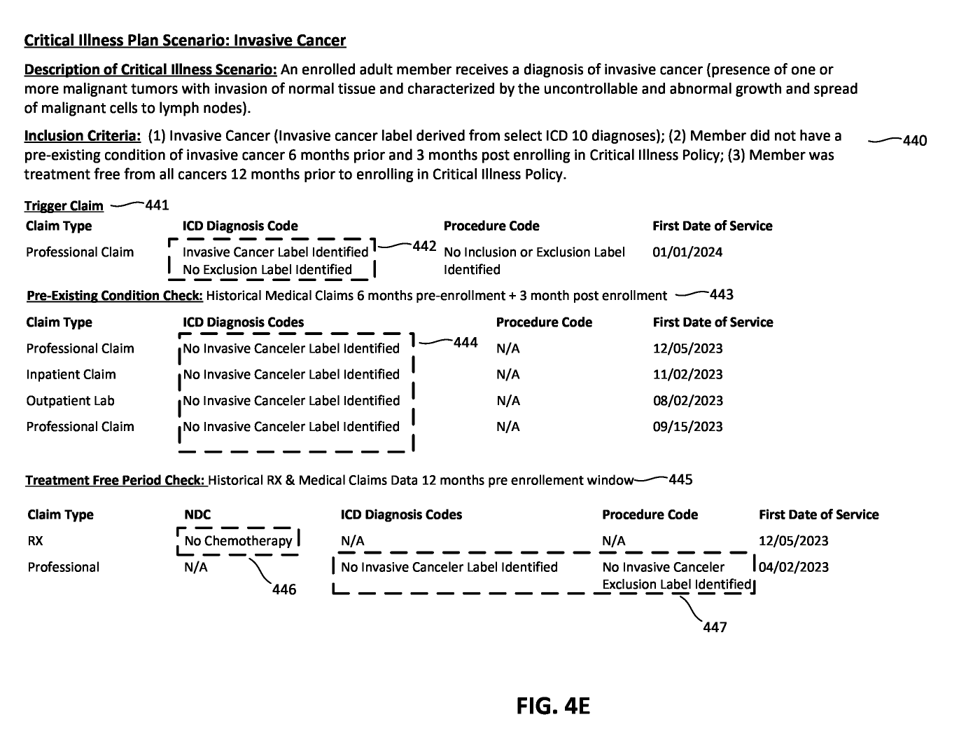

To implement this feature, the reimbursement opportunity identification unit 150 utilizes one or more medical crosswalks which provides a mapping of equivalent or near-equivalent codes between two different medical code sets. For example, the reimbursement opportunity identification unit 150 can utilize a crosswalk that maps medical codes from ICD-10-PCS (International Classification of Diseases, 10^{th} Revision, Procedure Coding System) medical codes to CPT (Current Procedural Terminology) medical codes to facilitate identification of equivalent codes in the medical claims and/or pharmaceutical claims information associated with a medical encounter. This is just one example of such a crosswalk that can be utilized by the reimbursement opportunity identification unit 150. Other crosswalks can be utilized instead of or in addition to this example. The reimbursement opportunity identification unit 150 the compares the medical codes on the included in the medical claims and/or pharmaceutical claims associated with each of the medical encounters with the reimbursement opportunity criteria to determine whether any of the reimbursement criteria opportunities have been satisfied. The reimbursement opportunity identification unit 150 uses the mapping information to determine whether the medical codes included on the medical claims and/or pharmaceutical claims satisfy the inclusion criteria and/or the exclusion criteria where there is not an exact match between the medical codes included in the medical claims and/or pharmaceutical claims of a medical encounter and medical codes used in the inclusion criteria and/or the exclusion criteria to identify equivalencies between the medical codes included in the medical claims and/or pharmaceutical claims and the reimbursement opportunity criteria. FIGS. 4A-4E, which are discussed in detail below, show some examples of how the reimbursement opportunity identification unit 150 determines whether the medical claims and/or pharmaceutical claims associated with a medical encounter satisfy the reimbursement opportunity criteria stored in the reimbursement opportunity criteria repository 145. The reimbursement opportunity identification unit 150 outputs the reimbursement opportunity information as reimbursement opportunity data structures which can be stored in the reimbursement opportunity information repository 130.

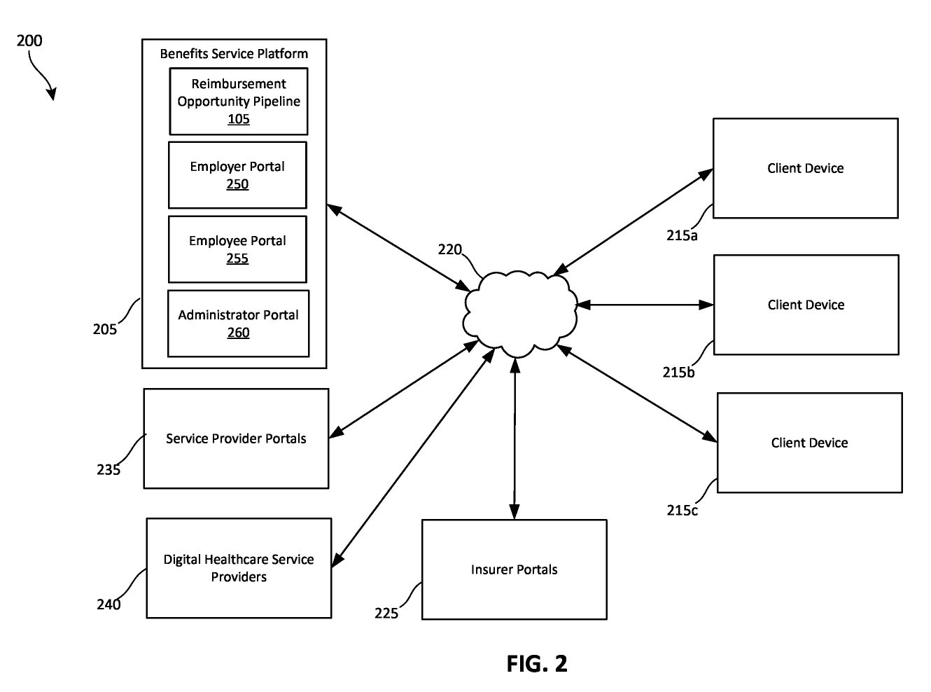

FIG. 2 is a diagram showing an example computing environment 200 that includes a benefits service platform 205 that utilizes the reimbursement opportunity pipeline shown in FIG. 1A. The benefits service platform 205 is a cloud-based service that provides tools for employers to manage benefits provided to employees and tools for the employees to access and utilize these benefits. The network 220 provides a means for communication among the various elements of the computing environment 200. The network 220 can include one or more wired and/or wireless public networks, private networks, or a combination thereof. The network 220 may be implemented at least in part by the Internet.

The benefits service platform 205 provides an employer portal 250 that provides a dashboard user interface that enables employers to access information associated with the insurance plans and benefits provided to their employees. These benefits may include insurance policy information as well as information about service providers with which the employer may have contracted to provide services to the employee. These services may include services provided by one or more digital healthcare service providers. The employer portal 250 provides user interfaces for viewing, accessing, or modifying the services that the employer has obtained on behalf of their employees. The employer may cover all or part of the cost associated with the services provided by the digital healthcare service providers. The employer portal 250 includes a search interface for the employer to search for insurers and/or digital healthcare service providers and obtain insurance plan information and/or service plan information from the insurers and/or digital healthcare service providers. The employer portal 250 provides tools that guide the employer through setting up new insurance coverage and/or a new service plan with an insurer and/or a digital health service provider.

The employee portal 255 provides a dashboard user interface that enables employees to access information associated with the benefits provided by their employer, information about insurance claims and prescriptions for the user and/or others covered by the user’s insurance, information on benefits utilization and how the user may save money by adjusting how they utilize their benefits. These recommendations can include recommendations, also referred to herein as “nudges,” that suggest that the user may utilize services provided by one or more digital health services of the digital healthcare service providers 240. These recommendations can identify medical and/or pharmaceutical claims that were not reimbursed or only reimbursed in part by the primary insurance of the employee, but the reimbursement opportunity pipeline 105 has determined that these claims should be covered by one or more supplemental health insurance plans of the employee. The employee portal 255 can provide reminders or nudges to the employee when such claims are identified by the reimbursement opportunity pipeline 105. The employee portal 255 can also provide tools that guide the user through submitting such claims to the insurers providing the supplemental health insurance plans for reimbursement. The employee portal 255 also includes tools that enable the employee to provide consent for the reimbursement opportunity pipeline 105 automatically analyze the employee’s medical and/or pharmaceutical claims to attempt to identify reimbursement opportunities.

The administrator portal 260 provides a dashboard user interface that enables administrators and/or other authorized users to configure features of the benefits service platform 205. The administrator portal 260 can provide user interfaces that enable the administrators to add new employers to the platform, remove existing employers from the platform, configure the tools available to existing employers, and/or perform other administrative actions on the benefits service platform 205. These actions can include configuring the reimbursement opportunity pipeline 105.

The service provider portals 235 provide tools that enable medical service providers, such as but not limited to doctors, dentists, optometrists, pharmacies, and/or other medical professionals to submit medical and/or pharmaceutical claims to the insurers on behalf of an insured user. The service provider portals 235 can include tools for providers to check on the status of a claim with an insurer. The service provider portals 235 may also permit the providers to amend and/or resubmit claims.

The digital healthcare service providers 240 provide digital healthcare point solutions. The digital healthcare service providers 240 can contract with employers to provide their services to employees of the employers. The digital healthcare service providers 240 can also provide these services to individual users requiring such services. The digital healthcare service providers 240 can provide a virtual clinic that users can access from a smart phone, tablet, computer, or other such client devices. The digital healthcare service providers 240 may provide various types of telemedicine services, counseling, and guidance to users. This approach enables the digital healthcare service providers 240 to provide high quality care to users regardless of the geographical location of the providers and the users. Consequently, the digital healthcare service providers 240 may provide such services at a significantly lower cost than traditional healthcare providers, which bear the significant cost of brick-and-mortar physical locations for their offices and/or clinics. The digital healthcare service providers 240 can submit medical and/or pharmaceutical claims to the insurance policy or polices of an insured employee.

The insurer portals 225 provide an interface for accessing insurance policy information for primary health insurance policies, supplemental health insurance policies, and/or other types of insurance policies provided by the insurers. The insurer portals 225 can provide tools that enable insured users to access policy information, make policy payments, obtain new policies, submit claims on existing policies, and/or perform other actions related to the managing the insured’s insurance. In some instances, the employer subsidizes all or a least a portion of the cost of the insurance and is responsible for making policy payments. The insurer portals 225 can also provide an interface that enables the reimbursement opportunity pipeline 105, the benefits service platform 205, and/or other benefits management tools to access the policy information for insured users that have provided consent to access their policy information and/or to permit the reimbursement opportunity pipeline 105, the benefits service platform 205, and/or other benefits management tools to submit claims on behalf of the insured users. The insurer portals 225 can also provide an interface that enables the reimbursement opportunity pipeline 105 and/or other components of the benefits service platform 205 to access electronic policy information for insured users, download the electronic policy information, map the electronic policy information to a data format according to the standard scheme utilized by the reimbursement opportunity pipeline 105 and/or other components of the benefits service platform 205, and to store the formatted policy information in the insurance plan information repository 110.

The client devices 215a, 215b, and 215c are computing devices that can be used to access the services provided by the benefits service platform 205, the insurer portals 225, the service provider portals 235, and/or the digital healthcare service providers 240. The client devices 215a, 215b, and 215c can have various form factors. For instance, the client devices 215a, 215b, and 215c can be implemented as a portable electronic device, such as a mobile phone, a tablet computer, a laptop computer, a portable digital assistant device, a portable game console, and/or other such devices. The client devices 215a, 215b, and 215c can also be implemented in computing devices having other form factors, such as a desktop computer, vehicle onboard computing system, a kiosk, a point-of-sale system, a video game console, and/or other types of computing devices. While the example implementation illustrated in FIG. 2 includes three client devices, other implementations may include a different number of client devices.

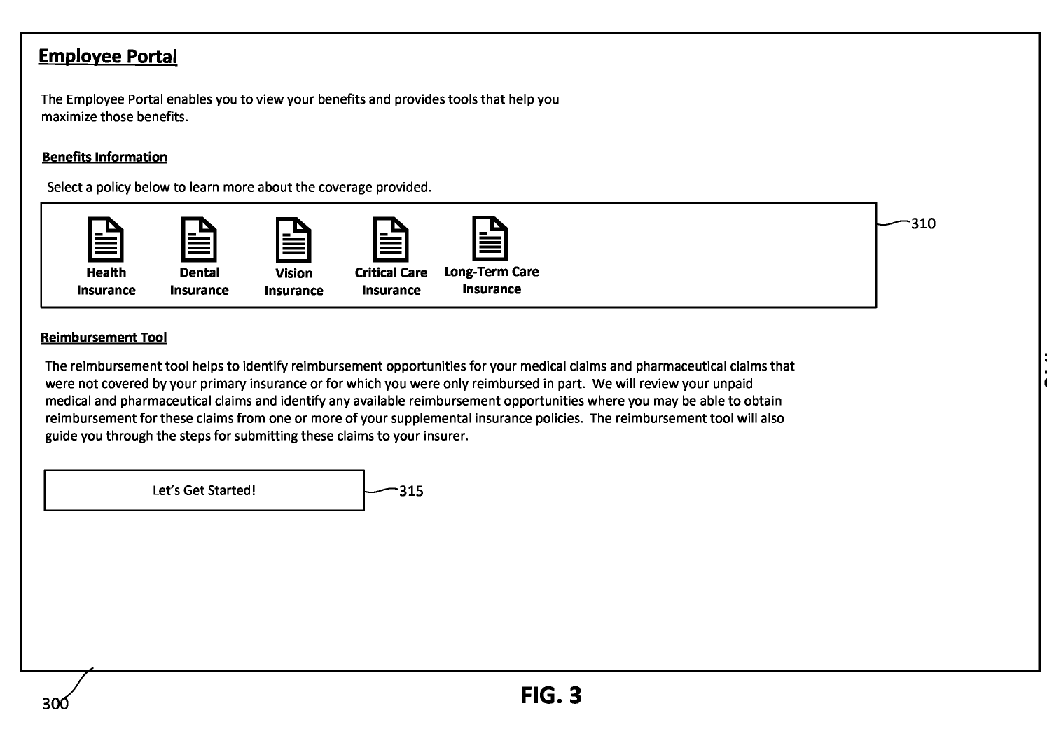

FIG. 3 is a diagram of an example user interface 300 of the employee portal 255 shown in FIG. 2. The user interface 300 includes a benefits pane 310 that includes an icon representing each of the primary and supplementary health care policies of the insured user. The user can click on, touch, or otherwise activate one of these icons to cause the user interface 300 to display information about that insurance policy. The user interface also includes a control 315, which when activated, causes the employee portal 255 to launch a reimbursement tool that utilizes the reimbursement opportunity pipeline 105 to analyze medical and/or pharmaceutical claims information and supplemental insurance policy information for the insured user to attempt to identify reimbursement opportunities and present these reimbursement opportunities to the insured user.

FIGS. 4A, 4B, 4C, 4D, and 4E are diagrams showing example reimbursement opportunities according to the techniques herein. FIGS. 4A, 4B, 4C, 4D, and 4E provide examples of claims associated with medical encounters that are associated with either accepted reimbursement opportunities or rejected reimbursement opportunities. As discussed in the preceding examples, the reimbursement opportunity criteria can be grouped into logical groupings referred to herein as reimbursement opportunity labels by the reimbursement opportunity criteria identification unit 140. Each reimbursement opportunity label is associated with one or more inclusion criteria and may also include one or more exclusion criteria. The reimbursement opportunity pipeline 105 determines that a reimbursement opportunity is likely to be accepted by an insurer providing a supplemental insurance policy when all of the inclusion criteria associated with a medical event have been satisfied and no exclusion criteria associated with the medical event have been satisfied. The inclusion criteria indicate one or more medical codes are required to be included on one or more of the medical claims or pharmaceutical claims associated with a respective medical encounter. The exclusion criteria indicate one or more medical codes, which if included on a claim associated with a medical encounter, will result in the insurer refusing to reimburse the medical and/or pharmaceutical claims associated with the medical encounter. In a non-limiting example, a supplemental insurance policy covers accidental injury excludes accidents resulting from uncovered activities that are deemed to be unsafe by the insurer, such as sky diving or scuba diving. In another non-limiting example, the exclusion criteria associated with the supplemental insurance policy are associated with negligent activities undertaken by the user, such as injuries resulting from the insured driving under the influence of drugs or alcohol. These examples are not exhaustive and non-limiting. Other policies may include different types of exclusion criteria.

The example shown in FIG. 4A shows an example of a reimbursement opportunity associated with reimbursement opportunity criteria 401 which reimburse the insured user for medical claims and/or pharmaceutical claims associated with an arm fracture resulting in surgery. The reimbursement opportunity criteria 401 is associated with two inclusion criteria that must be met by one or more medical and/or pharmaceutical claims associated with a medical event: (1) at least one of the claims must include an upper arm fracture diagnosis, and (2) at least one of the claims must include an open reduction surgery procedure of the upper arm. In this example, there are two claims associated with the medical encounter, claim 402 and claim 403. Individually, these claims would not satisfy the requirements of the inclusion criteria. Claim 402 includes the required procedure code 404 associated with the open reduction surgery procedure and claim 403 includes the required diagnosis code 405 for an upper arm fracture diagnosis. As discussed in the preceding examples, the specific diagnosis codes, procedure codes, and/or other medical codes associated with the claims can be mapped to identify equivalents across a plurality of code types. A technical advantage of this approach is that this approach significantly improves the ability of the reimbursement opportunity pipeline 105 to identify reimbursement opportunities in situations where the medical and/or pharmaceutical providers utilizes different types of medical codes. Utilizing this mapping enables the reimbursement opportunity pipeline 105 to automatically identify reimbursement opportunities that would have otherwise been overlooked if the medial and/or pharmaceutical claims did not include specific medical codes specified by the inclusion criteria. When grouped together as single medical encounter by the reimbursement opportunity pipeline 105, the claims 402 and 403 meet the inclusion criteria for the reimbursement opportunity. The reimbursement opportunity pipeline 105 can notify the insured user that their supplemental health plan will reimburse at least a portion of the costs associated with claims 402 and 403. The reimbursement opportunity pipeline 105 can automatically submit the claims to the insurer for reimbursement under an auto-adjudication process in some implementations if user consent has been provided. Consequently, the reimbursement opportunity pipeline 105 is able to assist the insured user in obtaining reimbursement for medical claims that otherwise would not have been eligible for reimbursement by the supplemental health care insurance.

The example shown in FIG. 4B shows an example of a reimbursement opportunity associated with reimbursement opportunity criteria 411. The reimbursement opportunity criteria 411 includes the same two inclusion criteria as the reimbursement opportunity criteria 411 shown in FIG. 4A and an exclusion criterion for accidents caused by driving while intoxicated. While the example shown in FIG. 4B includes a single exclusion criterion, the reimbursement opportunity criteria can include more than one exclusion criteria where the supplemental insurance policy of the insured user specifies multiple exclusion criteria. In the example shown in FIG. 4B, there are two claims associated with the medical encounter, claim 412 and claim 413. Claims 412 and 413 are similar to the claims 402 and 403 shown in FIG. 4A. Claim 412 includes the required procedure code 414 associated with the open reduction surgery procedure and claim 413 includes the required diagnosis code 415 for an upper arm fracture diagnosis. However, claim 412 includes a diagnosis code 416 that includes an accident exclusion code that indicates that the insured user was injured due to involvement in a vehicular accident in which the insured user was intoxicated. The exclusion code results in the reimbursement opportunity being rejected because the exclusion criterion is satisfied. This approach provides a technical solution associated with the technical problem of identifying valid reimbursement opportunities. A technical advantage of this approach is that the reimbursement opportunity pipeline 105 automatically identifies claims associated with a medical encounter that will be rejected due to the exclusion criteria thereby conserving computing resources that would have been used to submit the claims to the insurer that would ultimately be rejected as being ineligible for reimbursement.

The example shown in FIG. 4C shows a reimbursement opportunity associated with reimbursement opportunity criteria 420. In this example accident scenario, the insured user was injured by falling off a ladder and fractured their skull. The insured user is admitted to and stays at the hospital for three days in a non-intensive care unit (ICU) setting to treat their injury. The reimbursement opportunity criteria 420 is associated with four inclusion criteria and two exclusion criteria. The inclusion criteria include: (1) an accident occurred (the accident label is based on diagnosis codes associated with claims), (2) a hospitalization of one or more days, (3) a non-observation room stay at the hospital, and (4) a non-ICU hospital stay. The inclusion code in the present example includes an accident label, such as the accident label discussed in the preceding examples. The accident is a reimbursement opportunity label that includes one or more diagnosis codes, procedure codes, and/or other medical codes, which if present on a medical claim and/or a pharmaceutical claim is indicative of the insured user having been injured in an accident. FIG. 4C shows that reimbursement opportunity labels can be nested to create additional reimbursement opportunity labels. To create such nested labels, a reimbursement opportunity labels can be combined with one or more other reimbursement opportunity labels and/or with other reimbursement criteria to create new reimbursement opportunities. For instance, the accident label can be nested in the accidental injury and non-ICU hospitalization reimbursement opportunity criteria defined in this example. A technical advantage of this approach is it promotes label reuse and facilitates construction of new labels based on existing labels.

The reimbursement opportunity pipeline 105 has identified four medical claims that the reimbursement opportunity pipeline 105 has identified as being associated with this medical event: claim 422, claim 423, claim 424, and claim 425. Claim 422 is associated with a diagnosis code 426 that is one of a set preselected diagnosis codes that are associated with the accident label, indicating that the accident label inclusion criteria has been satisfied.

Claim 422 is also associated with a type of bill indicator 429 that indicates that the insured user was provided with inpatient care, and additional information 421 that indicates that the length of stay, and type of stay satisfy the inclusion criteria of the reimbursement opportunity criteria 420. Claim 423 also includes a diagnosis code 427 that is one of a set preselected diagnosis codes that are associated with the accident label. Furthermore, claim 424 includes a diagnosis code 428 that is one of a set preselected diagnosis codes that are associated with the accident label. Claim 425 is not associated with any medical codes that satisfy an inclusion or exclusion code. When grouped together as single medical encounter by the reimbursement opportunity pipeline 105, the claims 422, 423, 423, and 425 satisfy the inclusion criteria for the reimbursement opportunity criteria 420. The claims 422, 423, 423, and 425 are not associated with any exclusion criteria that would preclude reimbursement by the insurer providing the supplemental medical insurance policy in this example.

The example shown in FIG. 4D shows a reimbursement opportunity associated with reimbursement opportunity criteria 430. In this example accident scenario, the insured user received a prosthetic device for their leg after sustaining an injury in an accident earlier that year that resulted in amputation of their leg. The reimbursement opportunity criteria 430 is associated with three inclusion criteria: (1) the insured user receives a prosthetic device, (2) an accident occurred within 365 days prior to receipt of the prosthetic device, and (3) the insured member is at least 18 years old. The inclusion criteria utilizes the accident label discussed with respect to the example shown in FIG. 4C but adds a date restriction that indicates that the accident must have occurred up to one year prior to the insured user receiving the prosthetic device. The reimbursement opportunity pipeline 105 performs a lookback check to determine whether there are any claims that satisfy the accident label inclusion criteria that indicate that the insured user was injured in an accident in the past year.

In this example, the reimbursement opportunity pipeline 105 detects the trigger claim 431, which includes a procedure code 432 that indicates that the insured user received a prosthetic device. Detecting this procedure code 432 triggers the reimbursement opportunity pipeline 105 to perform an accident lookback check 433 to determine whether the second inclusion criterion has been satisfied, and a member age check 435 to determine whether the third inclusion criterion has been satisfied. The accident lookback check 433 found five medical claims for the insured user in the 365 days prior to receipt of the prosthetic device and two of these clams are associated with diagnosis codes 434 that indicate that the insured user was injured in an accident which satisfies the second inclusion criterion. The member age check 435 determines that the age of the insured user 436 satisfies the third inclusion criterion. Therefore, the reimbursement opportunity pipeline 105 determines that the reimbursement opportunity is available in this example, because the inclusion criteria associated with the supplemental insurance policy of the insured user have been satisfied. The reimbursement opportunity pipeline 105 can automatically notify the user of the reimbursement opportunity, automatically submit the claims for payment by the insurer, and/or perform other actions in response to determine that the reimbursement opportunity is available.

The example shown in FIG. 4E shows a reimbursement opportunity associated with reimbursement opportunity criteria 440. In the scenario shown in FIG. 4E, the insured user received a diagnosis of invasive cancer. The reimbursement opportunity criteria 440 is associated with associated with three inclusion criteria: (1) the insured user has been diagnosed with invasive cancer (the invasive cancel label is determined based on a select set of diagnosis codes), (2) the insured user did not have a pre-existing condition for invasive cancer six months prior to the enrolling in the critical illness plan and three months after enrolling in the critical illness policy, and (3) the insured user was treatment free from all cancers twelve months prior to enrolling in the critical illness policy.

In this example, the reimbursement opportunity pipeline 105 detects the trigger claim 441, which includes a procedure code 442 that indicates that the insured user has invasive cancer. The procedure code 442 is one of a preselected set of diagnosis codes associated with the invasive cancer label. As discussed in the preceding examples, the reimbursement opportunity pipeline 105 maps the diagnosis codes, procedure codes, and/or other medical codes associated with the claims to identify equivalent across a plurality of code types. These codes can be grouped together as a label for medical events, such as but not limited to the invasive cancer diagnosis in the example shown in FIG. 4E.

Detecting the procedure code 442 triggers the reimbursement opportunity pipeline 105 to perform a pre-existing condition check 443 to determine whether the second inclusion criterion has been satisfied, and a treatment free period check 445 to determine whether the third inclusion criterion has been satisfied. The pre-existing condition check 443 determines whether the insured user had any medical claims and/or pharmaceutical claims that were indicative of the insured user being diagnosed with invasive cancer within the period six months prior to enrollment to three months post-enrollment. The treatment free period check 445 identifies medical claims and/or pharmaceutical claims indicative of treatment for invasive cancer in the twelve month period prior to enrollment. The pre-existing condition check 443 has identified four medical claims that include diagnosis codes 444 that are not included in the set of diagnosis codes associated with the invasive cancer label. The treatment free period check 445 identifies one pharmaceutical claim and one medical claim that fall within the twelve month period prior to enrollment. The prescription claim is not associated with a National Drug Code (NDC) 446 that is associated with any treatments for invasive cancer. The NDC is a unique 10-digit number that identifies human drugs in the United States. Other countries and/or regions may utilize other similar identifiers for drugs that can be used by the reimbursement opportunity pipeline 105. The medical claim is associated with diagnosis code and procedure code information 447 that also indicates that insured user was not being treated for invasive cancer. Therefore, the reimbursement opportunity pipeline 105 determines that all of the inclusion criteria have been satisfied. The reimbursement opportunity pipeline 105 can automatically notify the user of the reimbursement opportunity, automatically submit the claims for payment by the insurer, and/or perform other actions in response to determine that the reimbursement opportunity is available.

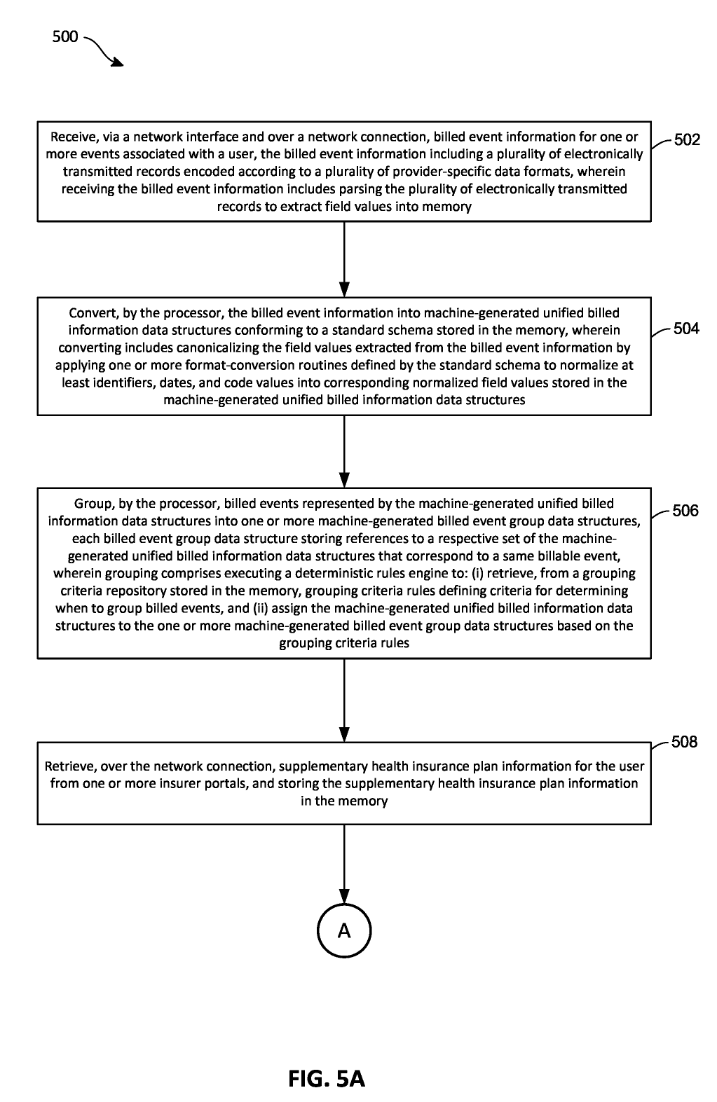

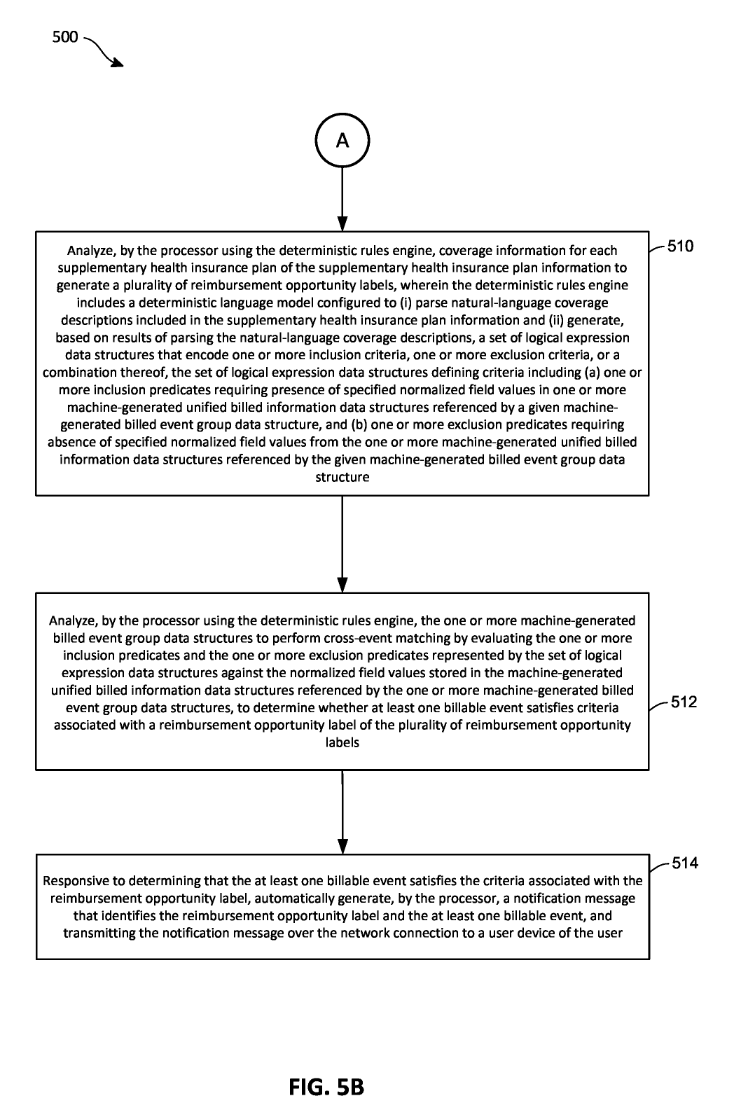

FIGS. 5A and 5B are a flow chart of another example process 500 for identifying reimbursement opportunities according to the techniques disclosed herein. The process 500 can be implemented by the reimbursement opportunity pipeline 105 as discussed in the preceding examples.

The process 500 includes an operation 502 of receiving, via a network interface and over a network connection, billed event information for one or more events associated with a user, the billed event information including a plurality of electronically transmitted records encoded according to a plurality of provider-specific data formats, wherein receiving the billed event information includes parsing the plurality of electronically transmitted records to extract field values into memory.

The process 500 includes an operation 504 of converting, by the processor, the billed event information into machine-generated unified billed information data structures conforming to a standard schema stored in the memory, wherein converting includes canonicalizing the field values extracted from the billed event information by applying one or more format-conversion routines defined by the standard schema to normalize at least identifiers, dates, and code values into corresponding normalized field values stored in the machine-generated unified billed information data structures.

The process 500 includes an operation 506 of grouping, by the processor, billed events represented by the machine-generated unified billed information data structures into one or more machine-generated billed event group data structures, each billed event group data structure storing references to a respective set of the machine-generated unified billed information data structures that correspond to a same billable event, wherein grouping comprises executing a deterministic rules engine to: (i) retrieve, from a grouping criteria repository stored in the memory, grouping criteria rules defining criteria for determining when to group billed events, and (ii) assign the machine-generated unified billed information data structures to the one or more machine-generated billed event group data structures based on the grouping criteria rules.

The process 500 includes an operation 508 of retrieving, over the network connection, supplementary health insurance plan information for the user from one or more insurer portals and storing the supplementary health insurance plan information in the memory.

The process 500 includes an operation 510 of analyzing, by the processor using the deterministic rules engine, coverage information for each supplementary health insurance plan of the supplementary health insurance plan information to generate a plurality of reimbursement opportunity labels, wherein the deterministic rules engine includes a deterministic language model configured to (i) parse natural-language coverage descriptions included in the supplementary health insurance plan information and (ii) generate, based on results of parsing the natural-language coverage descriptions, a set of logical expression data structures that encode one or more inclusion criteria, one or more exclusion criteria, or a combination thereof. The set of logical expression data structures defines criteria including (a) one or more inclusion predicates requiring presence of specified normalized field values in one or more machine-generated unified billed information data structures referenced by a given machine-generated billed event group data structure, and (b) one or more exclusion predicates requiring absence of specified normalized field values from the one or more machine-generated unified billed information data structures referenced by the given machine-generated billed event group data structure.

The process 500 includes an operation 512 of analyzing, by the processor using the deterministic rules engine 125, the one or more machine-generated billed event group data structures to perform cross-event matching by evaluating the one or more inclusion predicates and the one or more exclusion predicates represented by the set of logical expression data structures against the normalized field values stored in the machine-generated unified billed information data structures referenced by the one or more machine-generated billed event group data structures, to determine whether at least one billable event satisfies criteria associated with a reimbursement opportunity label of the plurality of reimbursement opportunity labels.

The process 500 includes an operation 514 of responsive to determining that the at least one billable event satisfies the criteria associated with the reimbursement opportunity label, automatically generating, by the processor, a notification message that identifies the reimbursement opportunity label and the at least one billable event, and transmitting the notification message over the network connection to a user device of the user. The notification unit 137 of the reimbursement opportunity pipeline 105. The reimbursement opportunity pipeline 105 also perform other operations, such as but not limited to automatically submitting claims to the insurer for reimbursement.

The detailed examples of systems, devices, and techniques described in connection with FIGS. 1A-5B are presented herein for illustration of the disclosure and its benefits. Such examples of use should not be construed to be limitations on the logical process embodiments of the disclosure, nor should variations of user interface methods from those described herein be considered outside the scope of the present disclosure. It is understood that references to displaying or presenting an item (such as, but not limited to, presenting an image on a display device, presenting audio via one or more loudspeakers, and/or vibrating a device) include issuing instructions, commands, and/or signals causing, or reasonably expected to cause, a device or system to display or present the item. In some embodiments, various features described in FIGS. 1A-5B are implemented in respective modules, which may also be referred to as, and/or include, logic, components, units, and/or mechanisms. Modules may constitute either software modules (for example, code embodied on a machine-readable medium) or hardware modules.

In some examples, a hardware module may be implemented mechanically, electronically, or with any suitable combination thereof. For example, a hardware module may include dedicated circuitry or logic that is configured to perform certain operations. For example, a hardware module may include a special-purpose processor, such as a field-programmable gate array (FPGA) or an Application Specific Integrated Circuit (ASIC). A hardware module may also include programmable logic or circuitry that is temporarily configured by software to perform certain operations and may include a portion of machine-readable medium data and/or instructions for such configuration. For example, a hardware module may include software encompassed within a programmable processor configured to execute a set of software instructions. It will be appreciated that the decision to implement a hardware module mechanically, in dedicated and permanently configured circuitry, or in temporarily configured circuitry (for example, configured by software) may be driven by cost, time, support, and engineering considerations.

Accordingly, the phrase “hardware module” should be understood to encompass a tangible entity capable of performing certain operations and may be configured or arranged in a certain physical manner, be that an entity that is physically constructed, permanently configured (for example, hardwired), and/or temporarily configured (for example, programmed) to operate in a certain manner or to perform certain operations described herein. As used herein, “hardware-implemented module” refers to a hardware module. Considering examples in which hardware modules are temporarily configured (for example, programmed), each of the hardware modules need not be configured or instantiated at any one instance in time. For example, where a hardware module includes a programmable processor configured by software to become a special-purpose processor, the programmable processor may be configured as respectively different special-purpose processors (for example, including different hardware modules) at different times. Software may accordingly configure a processor or processors, for example, to constitute a particular hardware module at one instance of time and to constitute a different hardware module at a different instance of time. A hardware module implemented using one or more processors may be referred to as being “processor implemented” or “computer implemented.”

Hardware modules can provide information to, and receive information from, other hardware modules. Accordingly, the described hardware modules may be regarded as being communicatively coupled. Where multiple hardware modules exist contemporaneously, communications may be achieved through signal transmission (for example, over appropriate circuits and buses) between or among two or more of the hardware modules. In embodiments in which multiple hardware modules are configured or instantiated at different times, communications between such hardware modules may be achieved, for example, through the storage and retrieval of information in memory devices to which the multiple hardware modules have access. For example, one hardware module may perform an operation and store the output in a memory device, and another hardware module may then access the memory device to retrieve and process the stored output.

In some examples, at least some of the operations of a method may be performed by one or more processors or processor-implemented modules. Moreover, the one or more processors may also operate to support performance of the relevant operations in a “cloud computing” environment or as a “software as a service” (SaaS). For example, at least some of the operations may be performed by, and/or among, multiple computers (as examples of machines including processors), with these operations being accessible via a network (for example, the Internet) and/or via one or more software interfaces (for example, an application program interface (API)). The performance of certain of the operations may be distributed among the processors, not only residing within a single machine, but deployed across several machines. Processors or processor-implemented modules may be in a single geographic location (for example, within a home or office environment, or a server farm), or may be distributed across multiple geographic locations.

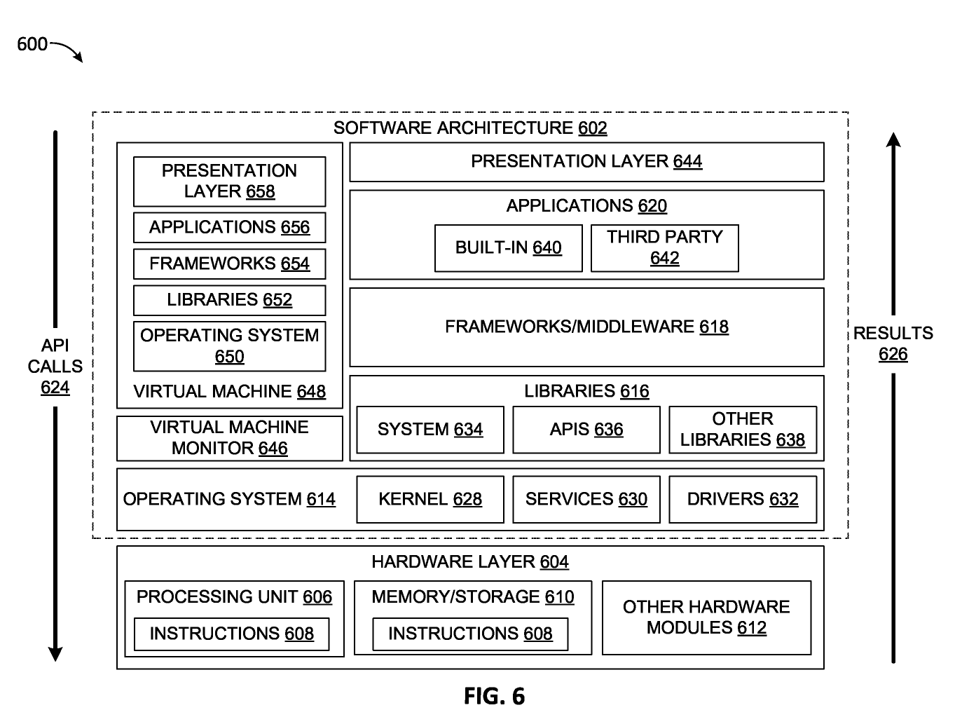

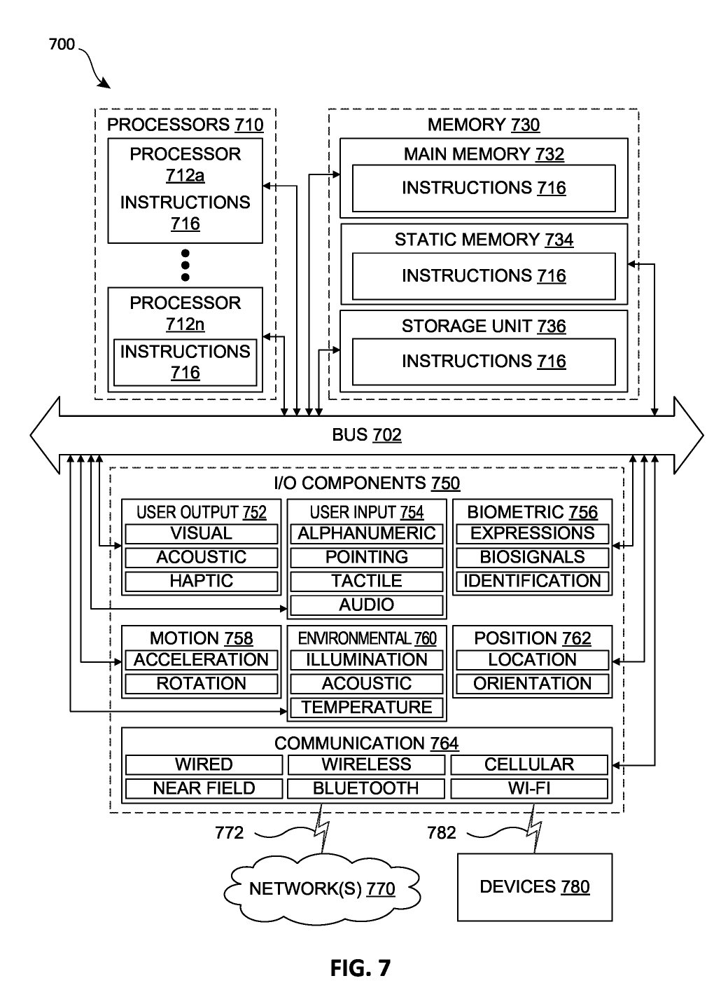

FIG. 6 is a block diagram 600 illustrating an example software architecture 602, various portions of which may be used in conjunction with various hardware architectures herein described, which may implement any of the above-described features. FIG. 6 is a non-limiting example of a software architecture, and it will be appreciated that many other architectures may be implemented to facilitate the functionality described herein. The software architecture 602 may execute on hardware such as a machine 700 of FIG. 7 that includes, among other things, processors 710, memory/storage, and input/output (I/O) components 750. A representative hardware layer 604 is illustrated and can represent, for example, the machine 700 of FIG. 7. The representative hardware layer 604 includes a processing unit 606 and associated executable instructions 608. The executable instructions 608 represent executable instructions of the software architecture 602, including implementation of the methods, modules and so forth described herein. The representative hardware layer 604 also includes a memory/storage 610, which also includes the executable instructions 608 and accompanying data. The representative hardware layer 604 may also include other hardware modules 612. Instructions 608 held by processing unit 606 may be portions of instructions 608 held by the memory/storage 610.

The example software architecture 602 may be conceptualized as layers, each providing various functionality. For example, the software architecture 602 may include layers and components such as an operating system (OS) 614, libraries 616, frameworks/middleware 618, applications 620, and a presentation layer 644. Operationally, the applications 620 and/or other components within the layers may invoke API calls 624 to other layers and receive corresponding results 626. The layers illustrated are representative in nature and other software architectures may include additional or different layers. For example, some mobile or special purpose operating systems may not provide the frameworks/middleware 618.

The OS 614 may manage hardware resources and provide common services. The OS 614 may include, for example, a kernel 628, services 630, and drivers 632. The kernel 628 may act as an abstraction layer between the representative hardware layer 604 and other software layers. For example, the kernel 628 may be responsible for memory management, processor management (for example, scheduling), component management, networking, security settings, and so on. The services 630 may provide other common services for the other software layers. The drivers 632 may be responsible for controlling or interfacing with the underlying, representative hardware layer 604. For instance, the drivers 632 may include display drivers, camera drivers, memory/storage drivers, peripheral device drivers (for example, via Universal Serial Bus (USB)), network and/or wireless communication drivers, audio drivers, and so forth depending on the hardware and/or software configuration.

The libraries 616 may provide a common infrastructure that may be used by the applications 620 and/or other components and/or layers. The libraries 616 typically provide functionality for use by other software modules to perform tasks, rather than interacting directly with the OS 614. The libraries 616 may include system libraries 634 (for example, C standard library) that may provide functions such as memory allocation, string manipulation, file operations. In addition, the libraries 616 may include API libraries 636 such as media libraries (for example, supporting presentation and manipulation of image, sound, and/or video data formats), graphics libraries (for example, an OpenGL library for rendering 2D and 3D graphics on a display), database libraries (for example, SQLite or other relational database functions), and web libraries (for example, WebKit that may provide web browsing functionality). The libraries 616 may also include a wide variety of other libraries 638 to provide many functions for applications 620 and other software modules.

The frameworks/middleware 618 provide a higher-level common infrastructure that may be used by the applications 620 and/or other software modules. For example, the frameworks/middleware 618 may provide various graphic user interface (GUI) functions, high-level resource management, or high-level location services. The frameworks/middleware 618 may provide a broad spectrum of other APIs for applications 620 and/or other software modules.

The applications 620 include built-in applications 640 and/or third-party applications 642. Examples of built-in applications 640 may include, but are not limited to, a contacts application, a browser application, a location application, a media application, a messaging application, and/or a game application. Third-party applications 642 may include any applications developed by an entity other than the vendor of the particular platform. The applications 620 may use functions available via OS 614, libraries 616, frameworks/middleware 618, and presentation layer 644 to create user interfaces to interact with users.

Some software architectures use virtual machines, as illustrated by a virtual machine 648. The virtual machine 648 provides an execution environment where applications/modules can execute as if they were executing on a hardware machine (such as the machine 700 of FIG. 7, for example). The virtual machine 648 may be hosted by a host OS (for example, OS 614) or hypervisor, and may have a virtual machine monitor 646 which manages operation of the virtual machine 648 and interoperation with the host operating system. A software architecture, which may be different from software architecture 602 outside of the virtual machine, executes within the virtual machine 648 such as an OS 650, libraries 652, frameworks 654, applications 656, and/or a presentation layer 658.

FIG. 7 is a block diagram illustrating components of an example implementation of a machine 700 configured to read instructions from a machine-readable medium (for example, a machine-readable storage medium) and perform any of the features described herein. The example machine 700 is in a form of a computer system, within which instructions 716 (for example, in the form of software components) for causing the machine 700 to perform any of the features described herein may be executed. As such, the instructions 716 may be used to implement modules or components described herein. The instructions 716 cause unprogrammed and/or unconfigured machine 700 to operate as a particular machine configured to carry out the described features. The machine 700 may be configured to operate as a standalone device or may be coupled (for example, networked) to other machines. In a networked deployment, the machine 700 may operate in the capacity of a server machine or a client machine in a server-client network environment, or as a node in a peer-to-peer or distributed network environment. Machine 700 may be embodied as, for example, a server computer, a client computer, a personal computer (PC), a tablet computer, a laptop computer, a netbook, a set-top box (STB), a gaming and/or entertainment system, a smart phone, a mobile device, a wearable device (for example, a smart watch), and an Internet of Things (IoT) device. Further, although only a single machine, machine 700, is illustrated, the term “machine” includes a collection of machines that individually or jointly execute the instructions 716.

The machine 700 may include processors 710, memory/storage 730, and I/O components 750, which may be communicatively coupled via, for example, a bus 702. The bus 702 may include multiple buses coupling various elements of machine 700 via various bus technologies and protocols. In an example, the processors 710 (including, for example, a central processing unit (CPU), a graphics processing unit (GPU), a digital signal processor (DSP), an ASIC, or a suitable combination thereof) may include one or more processors 712a to 712n that may execute the instructions 716 and process data. In some examples, one or more processors 710 may execute instructions provided or identified by one or more other processors 710. The term “processor” includes a multicore processor including cores that may execute instructions contemporaneously. Although FIG. 7 shows multiple processors, the machine 700 may include a single processor with a single core, a single processor with multiple cores (for example, a multicore processor), multiple processors each with a single core, multiple processors each with multiple cores, or any combination thereof. In some examples, the machine 700 may include multiple processors distributed among multiple machines.

The memory/storage 730 may include a main memory 732, a static memory 734, or other memory, and a storage unit 736, both accessible to the processors 710 such as via the bus 702. The storage unit 736 and main memory 732 and static memory 734 store the instructions 716 embodying any one or more of the functions described herein. The memory/storage 730 may also store temporary, intermediate, and/or long-term data for processors 710. The instructions 716 may also reside, completely or partially, within the main memory 732 and the static memory 734, within the storage unit 736, within at least one of the processors 710 (for example, within a command buffer or cache memory), within memory at least one of I/O components 750, or any suitable combination thereof, during execution thereof. Accordingly, the main memory 732, static memory 734, the storage unit 736, memory in processors 710, and memory in I/O components 750 are examples of machine-readable media.

As used herein, “machine-readable medium” refers to a device able to temporarily or permanently store instructions and data that cause machine 700 to operate in a specific fashion, and may include, but is not limited to, random-access memory (RAM), read-only memory (ROM), buffer memory, flash memory, optical storage media, magnetic storage media and devices, cache memory, network-accessible or cloud storage, other types of storage and/or any suitable combination thereof. The term “machine-readable medium” applies to a single medium, or combination of multiple media, used to store instructions (for example, instructions 716) for execution by a machine 700 such that the instructions, when executed by one or more processors 710 of the machine 700, cause the machine 700 to perform and one or more of the features described herein. Accordingly, a “machine-readable medium” may refer to a single storage device, as well as “cloud-based” storage systems or storage networks that include multiple storage apparatus or devices. The term “machine-readable medium” excludes signals per se.

The I/O components 750 may include a wide variety of hardware components adapted to receive input, provide output, produce output, transmit information, exchange information, capture measurements, and so on. The specific I/O components 750 included in a particular machine will depend on the type and/or function of the machine. For example, mobile devices such as mobile phones may include a touch input device, whereas a headless server or IoT device may not include such a touch input device. The particular examples of I/O components illustrated in FIG. 7 are in no way limiting, and other types of components may be included in machine 700. The grouping of I/O components 750 are merely for simplifying this discussion, and the grouping is in no way limiting. In various examples, the I/O components 750 may include user output components 752 and user input components 754. User output components 752 may include, for example, display components for displaying information (for example, a liquid crystal display (LCD) or a projector), acoustic components (for example, speakers), haptic components (for example, a vibratory motor or force-feedback device), and/or other signal generators. User input components 754 may include, for example, alphanumeric input components (for example, a keyboard or a touch screen), pointing components (for example, a mouse device, a touchpad, or another pointing instrument), and/or tactile input components (for example, a physical button or a touch screen that provides location and/or force of touches or touch gestures) configured for receiving various user inputs, such as user commands and/or selections.

In some examples, the I/O components 750 may include biometric components 756, motion components 758, environmental components 760, and/or position components 762, among a wide array of other physical sensor components. The biometric components 756 may include, for example, components to detect body expressions (for example, facial expressions, vocal expressions, hand or body gestures, or eye tracking), measure biosignals (for example, heart rate or brain waves), and identify a person (for example, via voice-, retina-, fingerprint-, and/or facial-based identification). The motion components 758 may include, for example, acceleration sensors (for example, an accelerometer) and rotation sensors (for example, a gyroscope). The environmental components 760 may include, for example, illumination sensors, temperature sensors, humidity sensors, pressure sensors (for example, a barometer), acoustic sensors (for example, a microphone used to detect ambient noise), proximity sensors (for example, infrared sensing of nearby objects), and/or other components that may provide indications, measurements, or signals corresponding to a surrounding physical environment. The position components 762 may include, for example, location sensors (for example, a Global Position System (GPS) receiver), altitude sensors (for example, an air pressure sensor from which altitude may be derived), and/or orientation sensors (for example, magnetometers).

The I/O components 750 may include communication components 764, implementing a wide variety of technologies operable to couple the machine 700 to network(s) 770 and/or device(s) 780 via respective communicative couplings 772 and 782. The communication components 764 may include one or more network interface components or other suitable devices to interface with the network(s) 770. The communication components 764 may include, for example, components adapted to provide wired communication, wireless communication, cellular communication, Near Field Communication (NFC), Bluetooth communication, Wi-Fi, and/or communication via other modalities. The device(s) 780 may include other machines or various peripheral devices (for example, coupled via USB).

In some examples, the communication components 764 may detect identifiers or include components adapted to detect identifiers. For example, the communication components 764 may include Radio Frequency Identification (RFID) tag readers, NFC detectors, optical sensors (for example, one- or multi-dimensional bar codes, or other optical codes), and/or acoustic detectors (for example, microphones to identify tagged audio signals). In some examples, location information may be determined based on information from the communication components 764, such as, but not limited to, geo-location via Internet Protocol (IP) address, location via Wi-Fi, cellular, NFC, Bluetooth, or other wireless station identification and/or signal triangulation.

In the preceding detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high level, without detail, in order to avoid unnecessarily obscuring aspects of the present teachings.

While various embodiments have been described, the description is intended to be exemplary, rather than limiting, and it is understood that many more embodiments and implementations are possible that are within the scope of the embodiments. Although many possible combinations of features are shown in the accompanying figures and discussed in this detailed description, many other combinations of the disclosed features are possible. Any feature of any embodiment may be used in combination with or substituted for any other feature or element in any other embodiment unless specifically restricted. Therefore, it will be understood that any of the features shown and/or discussed in the present disclosure may be implemented together in any suitable combination. Accordingly, the embodiments are not to be restricted except in light of the attached claims and their equivalents. Also, various modifications and changes may be made within the scope of the attached claims.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirements of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element. Furthermore, subsequent limitations referring back to “said element” or “the element” performing certain functions signifies that “said element” or “the element” alone or in combination with additional identical elements in the process, method, article, or apparatus are capable of performing all of the recited functions.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various examples for the purpose of streamlining the disclosure. This method of disclosure is not to be interpreted as reflecting an intention that the claims require more features than are expressly recited in each claim. Rather, as the following claims reflect, inventive subject matter lies in less than all features of a single disclosed example. Thus, the following claims are hereby incorporated into the Detailed Description, with each claim standing on its own as a separately claimed subject matter.

What is claimed is:

1. A data processing system comprising:
a processor; and
a memory storing executable instructions that, when executed by the processor, cause the data processing system to perform operations comprising:
receiving, via a network interface and over a network connection, billed event information for one or more events associated with a user, the billed event information including a plurality of electronically transmitted records encoded according to a plurality of provider-specific data formats, wherein receiving the billed event information includes parsing the plurality of electronically transmitted records to extract field values into memory;
converting, by the processor, the billed event information into machine-generated unified billed information data structures conforming to a standard schema stored in the memory, wherein converting includes canonicalizing the field values extracted from the billed event information by applying one or more format-conversion routines defined by the standard schema to normalize at least identifiers, dates, and code values into corresponding normalized field values stored in the machine-generated unified billed information data structures;
grouping, by the processor, billed events represented by the machine-generated unified billed information data structures into one or more machine-generated billed event group data structures, each billed event group data structure storing references to a respective set of the machine-generated unified billed information data structures that correspond to a same billable event, wherein grouping comprises executing a deterministic rules engine to: (i) retrieve, from a grouping criteria repository stored in the memory, grouping criteria rules defining criteria for determining when to group billed events, and (ii) assign the machine-generated unified billed information data structures to the one or more machine-generated billed event group data structures based on the grouping criteria rules;
retrieving, over the network connection, supplementary health insurance plan information for the user from one or more insurer portals, and storing the supplementary health insurance plan information in the memory;
analyzing, by the processor using the deterministic rules engine, coverage information for each supplementary health insurance plan of the supplementary health insurance plan information to generate a plurality of reimbursement opportunity labels, wherein the deterministic rules engine includes a deterministic language model configured to (i) parse natural-language coverage descriptions included in the supplementary health insurance plan information and (ii) generate, based on results of parsing the natural-language coverage descriptions, a set of logical expression data structures that encode one or more inclusion criteria, one or more exclusion criteria, or a combination thereof, the set of logical expression data structures defining criteria including (a) one or more inclusion predicates requiring presence of specified normalized field values in one or more machine-generated unified billed information data structures referenced by a given machine-generated billed event group data structure, and (b) one or more exclusion predicates requiring absence of specified normalized field values from the one or more machine-generated unified billed information data structures referenced by the given machine-generated billed event group data structure;
analyzing, by the processor using the deterministic rules engine, the one or more machine-generated billed event group data structures to perform cross-event matching by evaluating the one or more inclusion predicates and the one or more exclusion predicates represented by the set of logical expression data structures against the normalized field values stored in the machine-generated unified billed information data structures referenced by the one or more machine-generated billed event group data structures, to determine whether at least one billable event satisfies criteria associated with a reimbursement opportunity label of the plurality of reimbursement opportunity labels; and
responsive to determining that the at least one billable event satisfies the criteria associated with the reimbursement opportunity label, automatically generating, by the processor, a notification message that identifies the reimbursement opportunity label and the at least one billable event, and transmitting the notification message over the network connection to a user device of the user.

2. The data processing system of claim 1, wherein the machine-generated unified billed information data structures includes normalized fields for one or more of medical codes, dates of service, provider identifiers, and reimbursement attributes.

3. The data processing system of claim 1, wherein the memory further stores executable instructions that, when executed, cause the processor alone or in combination with other processors to perform operations of:
retrieving, over the network connection, supplementary health insurance plan information for the user from one or more insurer portal; and
analyzing coverage information for each supplementary health insurance plan of the supplementary health insurance plan information using the deterministic rules engine to generate the plurality of reimbursement opportunity labels.

4. The data processing system of claim 3, wherein to analyze the coverage information for each supplementary health insurance plan of the supplementary health insurance plan information to generate the plurality of reimbursement opportunity labels, the memory further stores executable instructions that, when executed, cause the processor alone or in combination with other processors to perform operations of:
mapping one or more medical codes associated with the one or more inclusion criteria to one or more equivalent medical codes using one or more crosswalks.

5. The data processing system of claim 4, wherein the memory further stores executable instructions that, when executed, cause the processor alone or in combination with other processors to perform operations of:
determining, using the deterministic rules engine, whether the one or more inclusion criteria has been satisfied based on the one or more medical codes associated with the one or more inclusion criteria and the one or more equivalent medical codes.

6. The data processing system of claim 4, wherein to analyze the coverage information for each supplementary health insurance plan of the supplementary health insurance plan information to generate the plurality of reimbursement opportunity labels, the memory further stores executable instructions that, when executed, cause the processor alone or in combination with other processors to perform operations of:
mapping one or more medical codes associated with the one or more exclusion criteria to one or more equivalent medical codes using one or more crosswalks.

7. The data processing system of claim 6, wherein the memory further stores executable instructions that, when executed, cause the processor alone or in combination with other processors to perform operations of:
determining, using the deterministic rules engine, whether the one or more exclusion criteria has been satisfied based on the one or more medical codes associated with the one or more exclusion criteria and the one or more equivalent medical codes.

8. A method implemented in a data processing system for identifying reimbursement opportunities, the method comprising:
receiving, via a network interface and over a network connection, billed event information for one or more events associated with a user, the billed event information including a plurality of electronically transmitted records encoded according to a plurality of provider-specific data formats, wherein receiving the billed event information includes parsing the plurality of electronically transmitted records to extract field values into memory;
converting, by a processor of the data processing system, the billed event information into machine-generated unified billed information data structures conforming to a standard schema stored in the memory, wherein converting includes canonicalizing the field values extracted from the billed event information by applying one or more format-conversion routines defined by the standard schema to normalize at least identifiers, dates, and code values into corresponding normalized field values stored in the machine-generated unified billed information data structures;
grouping, by the processor, billed events represented by the machine-generated unified billed information data structures into one or more machine-generated billed event group data structures, each billed event group data structure storing references to a respective set of the machine-generated unified billed information data structures that correspond to a same billable event, wherein grouping comprises executing a deterministic rules engine to: (i) retrieve, from a grouping criteria repository stored in the memory, grouping criteria rules defining criteria for determining when to group billed events, and (ii) assign the machine-generated unified billed information data structures to the one or more machine-generated billed event group data structures based on the grouping criteria rules;
retrieving, over the network connection, supplementary health insurance plan information for the user from one or more insurer portals, and storing the supplementary health insurance plan information in the memory;
analyzing, by the processor using the deterministic rules engine, coverage information for each supplementary health insurance plan of the supplementary health insurance plan information to generate a plurality of reimbursement opportunity labels, wherein the deterministic rules engine includes a deterministic language model configured to (i) parse natural-language coverage descriptions included in the supplementary health insurance plan information and (ii) generate, based on results of parsing the natural-language coverage descriptions, a set of logical expression data structures that encode one or more inclusion criteria, one or more exclusion criteria, or a combination thereof, the set of logical expression data structures defining criteria including (a) one or more inclusion predicates requiring presence of specified normalized field values in one or more machine-generated unified billed information data structures referenced by a given machine-generated billed event group data structure, and (b) one or more exclusion predicates requiring absence of specified normalized field values from the one or more machine-generated unified billed information data structures referenced by the given machine-generated billed event group data structure;
analyzing, by the processor using the deterministic rules engine, the one or more machine-generated billed event group data structures to perform cross-event matching by evaluating the one or more inclusion predicates and the one or more exclusion predicates represented by the set of logical expression data structures against the normalized field values stored in the machine-generated unified billed information data structures referenced by the one or more machine-generated billed event group data structures, to determine whether at least one billable event satisfies criteria associated with a reimbursement opportunity label of the plurality of reimbursement opportunity labels; and
responsive to determining that the at least one billable event satisfies the criteria associated with the reimbursement opportunity label, automatically generating, by the processor, a notification message that identifies the reimbursement opportunity label and the at least one billable event, and transmitting the notification message over the network connection to a user device of the user.

9. The method of claim 8, wherein the machine-generated unified billed information data structures includes normalized fields for one or more of medical codes, dates of service, provider identifiers, and reimbursement attributes.

10. The method of claim 8, further comprising:
retrieving, over the network connection, supplementary health insurance plan information for the user from one or more insurer portal; and
analyzing coverage information for each supplementary health insurance plan of the supplementary health insurance plan information using the deterministic rules engine to generate the plurality of reimbursement opportunity labels.

11. The method of claim 10, wherein analyzing the coverage information for each supplementary health insurance plan of the supplementary health insurance plan information to generate the plurality of reimbursement opportunity labels further comprises:
mapping one or more medical codes associated with the one or more inclusion criteria to one or more equivalent medical codes using one or more crosswalks.

12. The method of claim 11, further comprising:
determining, using the deterministic rules engine, whether the one or more inclusion criteria has been satisfied based on the one or more medical codes associated with the one or more inclusion criteria and the one or more equivalent medical codes.

13. The method of claim 11, wherein analyzing the coverage information for each supplementary health insurance plan of the supplementary health insurance plan information to generate the plurality of reimbursement opportunity labels further comprises:
mapping one or more medical codes associated with the one or more exclusion criteria to one or more equivalent medical codes using one or more crosswalks.

14. The method of claim 13, further comprising:
determining, using the deterministic rules engine, whether the one or more exclusion criteria has been satisfied based on the one or more medical codes associated with the one or more exclusion criteria and the one or more equivalent medical codes.

15. A machine-readable medium on which are stored instructions that, when executed, cause a processor alone or in combination with other processors to perform operations of:
receiving, via a network interface and over a network connection, billed event information for one or more events associated with a user, the billed event information including a plurality of electronically transmitted records encoded according to a plurality of provider-specific data formats, wherein receiving the billed event information includes parsing the plurality of electronically transmitted records to extract field values into memory;
converting, by the processor, the billed event information into machine-generated unified billed information data structures conforming to a standard schema stored in the memory, wherein converting includes canonicalizing the field values extracted from the billed event information by applying one or more format-conversion routines defined by the standard schema to normalize at least identifiers, dates, and code values into corresponding normalized field values stored in the machine-generated unified billed information data structures;
grouping, by the processor, billed events represented by the machine-generated unified billed information data structures into one or more machine-generated billed event group data structures, each billed event group data structure storing references to a respective set of the machine-generated unified billed information data structures that correspond to a same billable event, wherein grouping comprises executing a deterministic rules engine to: (i) retrieve, from a grouping criteria repository stored in the memory, grouping criteria rules defining criteria for determining when to group billed events, and (ii) assign the machine-generated unified billed information data structures to the one or more machine-generated billed event group data structures based on the grouping criteria rules;
retrieving, over the network connection, supplementary health insurance plan information for the user from one or more insurer portals, and storing the supplementary health insurance plan information in the memory;
analyzing, by the processor using the deterministic rules engine, coverage information for each supplementary health insurance plan of the supplementary health insurance plan information to generate a plurality of reimbursement opportunity labels, wherein the deterministic rules engine includes a deterministic language model configured to (i) parse natural-language coverage descriptions included in the supplementary health insurance plan information and (ii) generate, based on results of parsing the natural-language coverage descriptions, a set of logical expression data structures that encode one or more inclusion criteria, one or more exclusion criteria, or a combination thereof, the set of logical expression data structures defining criteria including (a) one or more inclusion predicates requiring presence of specified normalized field values in one or more machine-generated unified billed information data structures referenced by a given machine-generated billed event group data structure, and (b) one or more exclusion predicates requiring absence of specified normalized field values from the one or more machine-generated unified billed information data structures referenced by the given machine-generated billed event group data structure;
analyzing, by the processor using the deterministic rules engine, the one or more machine-generated billed event group data structures to perform cross-event matching by evaluating the one or more inclusion predicates and the one or more exclusion predicates represented by the set of logical expression data structures against the normalized field values stored in the machine-generated unified billed information data structures referenced by the one or more machine-generated billed event group data structures, to determine whether at least one billable event satisfies criteria associated with a reimbursement opportunity label of the plurality of reimbursement opportunity labels; and
responsive to determining that the at least one billable event satisfies the criteria associated with the reimbursement opportunity label, automatically generating, by the processor, a notification message that identifies the reimbursement opportunity label and the at least one billable event, and transmitting the notification message over the network connection to a user device of the user.

16. The machine-readable medium of claim 15, wherein the machine-generated unified billed information data structures includes normalized fields for one or more of medical codes, dates of service, provider identifiers, and reimbursement attributes.

17. The machine-readable medium of claim 15, wherein the machine-readable medium further stores executable instructions that, when executed, cause the processor alone or in combination with other processors to perform operations of:
retrieving, over the network connection, supplementary health insurance plan information for the user from one or more insurer portal; and
analyzing coverage information for each supplementary health insurance plan of the supplementary health insurance plan information using the deterministic rules engine to generate the plurality of reimbursement opportunity labels.

18. The machine-readable medium of claim 17, wherein to analyze the coverage information for each supplementary health insurance plan of the supplementary health insurance plan information to generate the plurality of reimbursement opportunity labels, the machine-readable medium further stores executable instructions that, when executed, cause the processor alone or in combination with other processors to perform operations of:
mapping one or more medical codes associated with the one or more inclusion criteria to one or more equivalent medical codes using one or more crosswalks.

19. The machine-readable medium of claim 18, wherein the machine-readable medium further stores executable instructions that, when executed, cause the processor alone or in combination with other processors to perform operations of:
determining, using the deterministic rules engine, whether the one or more inclusion criteria has been satisfied based on the one or more medical codes associated with the one or more inclusion criteria and the one or more equivalent medical codes.

20. The machine-readable medium of claim 18, wherein to analyze the coverage information for each supplementary health insurance plan of the supplementary health insurance plan information to generate the plurality of reimbursement opportunity labels, the machine-readable medium further stores executable instructions that, when executed, cause the processor alone or in combination with other processors to perform operations of:
mapping one or more medical codes associated with the one or more exclusion criteria to one or more equivalent medical codes using one or more crosswalks.
